(19) United States
(12) Patent Application Publication
(10) Pub. No.: US 2002/0119969 A1
(43) Pub. Date: Aug. 29, 2002

(54) Novel process

(76) Inventors: Soren Ebdrup, Copenhagen O (DK); Flemming Elmelund Nielsen, Virum (DK)

Correspondence Address: Reza Green, Esq., Novo Nordisk of North America Inc., Suite 6400, 405 Lexington Avenue, New York, NY, 10174-6401, US

(21) Appl. No.: 09/924,350

(22) Filed: Aug. 8, 2001

Related U.S. Application Data
(63) Continuation of application No. PCT/DK00/00343, filed on Jun. 26, 2000.
(60) Provisional application No. 60/142,759, filed on Jul. 8, 1999.

(30) Foreign Application Priority Data
Jun. 30, 1999 (DK) PA 1999 00938

(51) Int. Cl. A61K 31/549; C07D 285/34

(52) U.S. Cl. 514/222.8; 514/223.2; 544/10; 544/12

(57) ABSTRACT

The present invention relates to a novel processes for preparing pharmaceutically active fused 1,2,4-thiadiazine derivatives of general formula (I) as defined in the description and intermediates therefore.

### CROSS REFERENCE TO RELATED APPLICATIONS

This application is a continuation of PCT/DK00/00343 filed on Jun. 26, 2000, which claims priority under 35 U.S.C. 119 of Danish application no. PA 1999 00938 filed on Jun. 30, 1999, and U.S. provisional application No. 60/142,759 filed on Jul. 8, 1999, the contents of which are fully incorporated herein by reference.

The present invention relates to novel processes for preparing pharmaceutically active compounds and intermediates therefore.

Pharmaceutical active compounds acting as potent and selective potassium channel openers that, by inhibiting insulin release and inducing P-cell rest, can be used in treatment of Type I and Type II diabetes are described in PCT Publication WO 97/26265. The compounds listed on page 46, line 33 to page 47, line 15 in PCT Publication WO 97/26265 are preferred.

Ways of synthesizing those compounds are described in PCT Publication WO 97/26265 on pages 20 to 25 and in PCT Publication WO 99/03861.

The present invention provides alternative methods of synthesis for the above mentioned compounds in a more efficient way.

U.S. Pat. No. 5,459,138 discloses certain pyridothiadiazines and their preparation. However, the patent relates to the synthesis of pyridines only and contains no disclosure as regards the synthesis of the considerably less reactive 5 membered heterocyclic ring systems. However, as described in Advanced Organic Chemistry, Forth Edition, 1992, J. March, page 649, 2- and 4-chloropyridine are especially activated to nucleophilic aromatic substitution reactions. Further, in Organic Chemistry, Fourth Edition, 1983, Morrison and Boyd, page 1273, it is described that 5 membered heterocycles in general are activated to electrophilic aromatic substitution and deactivated towards nucleophilic aromatic substitution.

Thus, it has surprisingly been found that 5 membered heterocycles can undergo the reactions described in the above U.S. Pat. No. 5,459,138.

The present invention has been developed on the basis that compounds of formula (I) below are either valuable chemical intermediates useful for the manufacture of pharmaceutical active compounds, such as those compounds listed on page 46, line 33 to page 47, line 15 in PCT Publication WO 97/26265, or are themselves active compounds, such as disclosed in PCT Publication WO 97/26265.

The present invention provides novel processes for the preparation of fused 1,2,4-thiadiazine derivatives of the general formula (I):

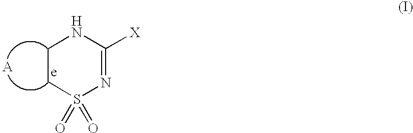

wherein

X is NR²R³, SR¹, S(═O)R¹, S(═O)₂R¹, or OR¹;

R¹ is hydrogen, C₃₋₆-cycloalkyl or (C₃₋₆-cycloalkyl)C₁₋₆-alkyl the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms, optionally being mono- or polysubstituted with halogen, cyano, trifluoromethyl, C₁₋₆-alkyl, C₁₋₆-alkoxy, C₁₋₆-alkoxy-C₁₋₆-alkyl, aryl, arylalkyl, hydroxy, oxo, nitro, amino, C₁₋₆-monoalkyl or dialkylamino; or straight or branched C₁₋₁₈-alkyl, C₂₋₁₈-alkenyl or C₂₋₁₈-alkynyl, each of the groups being optionally mono- or polysubstituted with halogen, hydroxy, C₁₋₆-alkoxy, C₁₋₆-alkylthio, C₃₋₆-cycloalkyl, nitro, amino, C₁₋₆-monoalkyl- or dialkylamino, cyano, oxo, formyl, acyl, carboxy, C₁₋₆-alkoxycarbonyl, carbamoyl, formylamino, C₁₋₆-alkylcarbonylamino, aryl, aryloxy, arylalkoxy; bicycloalkyl, aryl, heteroaryl, arylalkyl or heteroarylalkyl, each of the groups being optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;

R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or polysubstituted with halogen;

R³ is hydrogen, C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl; or

R³ is —OR⁴; —C(═Z)R⁴; —NR⁴R⁵; bicycloalkyl, aryl, heteroaryl, arylalkyl or heteroarylalkyl optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;

R⁴ is hydrogen; C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

Z is O or S;

R⁵ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or

when R³ is —NR⁴R⁵, R⁴ and R⁵ together with the nitrogen atom form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

when X is NR²R³, R² and R³ together with the nitrogen atom form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;

A together with the carbon atoms forming bond e of formula (I) represents a 5 membered heterocyclic system comprising one or more nitrogen-, oxygen- or sulfur atoms, the heterocyclic cyclic systems optionally being mono- or polysubstituted with halogen; C₁₋₁₈-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, aryl, arylalkyl, aryloxy, the aryl group optionally being mono- or polysubstituted with C₁₋₆-alkyl, perhalomethyl, halogen, hydroxy or C₁₋₆-alkoxy; C₁₋₆-alkoxycarbonyl; C₁₋₆-alkoxycarbonyl-C₁₋₆-alkyl; carbamyl; carbamylmethyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thiocarbamyl; thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; formyl; or a 5-6 membered nitrogen, oxygen or sulfur containing ring, optionally substituted with C₁₋₆-alkyl or phenyl, the phenyl group optionally being mono- or polysubstituted with C₁₋₆-alkyl, perhalomethyl, halogen, hydroxy or C₁₋₆-alkoxy; or

a salt thereof with a pharmaceutically acceptable acid or base.

Within its scope the invention the process for preparation of compounds of formula (I) includes all optical isomers of compounds of formula (I), some of which are optically active, and also their mixtures including racemic mixture thereof.

The scope of the invention also includes all tautomeric forms of the compounds of formula (I) as well as metabolites or prodrugs of a compound of formula (I).

The salts include pharmaceutically acceptable acid addition salts, pharmaceutically acceptable metal salts or optionally alkylated ammonium salts, such as hydrochloric, hydrobromic, hydroiodic, phosphoric, sulfuric, trifluoroacetic, trichloroacetic, oxalic, maleic, pyruvic, malonic, succinic, citric, tartaric, fumaric, mandelic, benzoic, cinnamic, methanesulfonic, ethanesulfonic, picric and the like, and include acids related to the pharmaceutically acceptable salts listed in Journal of Pharmaceutical Science, 66, 2 (1977) and incorporated herein by reference, or lithium, sodium, potassium, magnesium and the like.

A “metabolite” of a compound disclosed in this application is an active derivative of a compound disclosed herein which is produced when the compound is metabolized. Metabolites of compounds disclosed herein can be identified either by administration of a compound to a host and an analysis of blood samples from the host, or by incubation of compounds with hepatic cells in vitro and analysis of the incubant. A “prodrug” is a compound that either is converted into a compound disclosed in the application in vivo or has the same active metabolite as a compound disclosed in this application.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The terms “C₂₋₆-alkenyl” and “C₂₋₁₈-alkenyl” as used herein refers to an unsaturated hydrocarbon chain having 2-6 or 2-18 carbon atoms and one double bond such as e.g. vinyl, 1-propenyl, allyl, isopropenyl, n-butenyl, n-pentenyl and n-hexenyl.

The term “C₃₋₆-cycloalkyl” as used herein refers to a radical of a saturated cyclic hydrocarbon with the indicated number of carbons such as cyclopropyl, cyclobutyl, cyclopentyl, or cyclohexyl.

The terms “C₂₋₆-alkynyl” and “C₂₋₁₈-alkynyl” as used herein refers to unsaturated hydrocarbons which contain triple bonds, such as e.g. —C≡CH, —C≡CCH₃, —CH₂C≡CH, —CH₂CH₂C≡CH, —CH(CH₃)C≡CH, and the like.

The term “C₁₋₆-alkoxy-C₁₋₆-alkyl” as used herein refers to a group of 2-12 carbon atoms interrupted by an O such as e.g. CH₂—O—CH₃, CH₂—O—CH₂—CH₃, CH₂—O—CH(CH₃)₂ and the like.

The term “halogen” means fluorine, chlorine, bromine or iodine.

The term “perhalomethyl” means trifluoromethyl, trichloromethyl, tribromomethyl or triiodomethyl.

The terms “C₁₋₆-alkyl”, “C₁₋₁₂-alkyl” and “C₁₋₁₈-alkyl” as used herein, alone or in combination, refers to a straight or branched, saturated hydrocarbon chain having the indicated number of carbon atoms such as e.g. methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl, 1,2,2-trimethylpropyl and the like. The term “C₁₋₁₈-alkyl” as used herein also includes secondary C₃₋₆-alkyl and tertiary C₄₋₆-alkyl.

The term “C₁₋₆-monoalkylamino” as used herein refers to an amino group wherein one of the hydrogen atoms is substituted with a straight or branched, saturated hydrocarbon chain having the indicated number of carbon atoms such as e.g. methylamino, ethylamino, propylamino, n-butylamino, sec-butylamino, isobutylamino, tert-butylamino, n-pentylamino, 2-methylbutylamino, n-hexylamino, 4-methylpentylamino, neopentylamino, n-hexylamino, 2,2-dimethylpropylamino and the like.

The term “C₁₋₆-dialkylamino” as used herein refers to an amino group wherein the two hydrogen atoms independently are substituted with a straight or branched, saturated hydrocarbon chain having the indicated number of carbon atoms; such as dimethylamino, N-ethyl-N-methylamino, diethylamino, dipropylamino, N-(n-butyl)-N-methylamino, di(n-pentyl) amino, and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “3-12 membered mono- or bicyclic system” as used herein refers to a monovalent substituent of formula —NR²R³ or —NR⁸R⁹ where R² and R³, or R⁸ and R⁹ together with the nitrogen atom form a 3-12 membered mono- or bicyclic system, in which one or more of the carbon atoms may be exchanged with nitrogen, oxygen or sulfur, such as 1-pyrrolidyl, piperidino, morpholino, thiomorpholino, 4-methylpiperazin-1-yl, 7-azabicyclo[2.2.1]heptan-7-yl, tropanyl and the like.

The term “3-6 membered saturated ring system” as used herein refers to a monovalent substituent comprising a monocyclic saturated system containing one or more hetero atoms selected from nitrogen, oxygen and sulfur and having 3-6 members and having its free valence from a carbon atom, e.g. 2-pyrrolidyl, 4-piperidyl, 3-morpholinyl, 1,4-dioxan-2-yl, 5-oxazolidinyl, 4-isoxazolidinyl, or 2-thiomorpholinyl.

The term “bicycloalkyl” as used herein refers to a monovalent substituent comprising a bicyclic structure made of 6-12 carbon atoms such as e.g. 2-norbornyl, 7-norbornyl, 2-bicyclo[2.2.2]octyl, and 9-bicyclo[3.3.1]nonanyl.

The term “aryl” as used herein refers to phenyl, 1-naphthyl, or 2-naphthyl.

The term “heteroaryl” as used herein, alone or in combination, refers to a monovalent substituent comprising a 5-6 membered monocyclic aromatic system or a 9-10 membered bicyclic aromatic system containing one or more heteroatoms selected from nitrogen, oxygen and sulfur, e.g. pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine, and purine.

The term “arylalkyl” as used herein refers to a straight or branched saturated carbon chain containing from 1 to 6 carbons substituted with an aromatic carbohydride; such as benzyl, phenethyl, 3-phenylpropyl, 1-naphtylmethyl, 2-(1-naphtyl)ethyl and the like.

The term “aryloxy” as used herein refers to phenoxy, 1-naphthyloxy or 2-naphthyloxy.

The term “arylalkoxy” as used herein refers to a C₁₋₆-alkoxy group substituted with an aromatic carbohydride, such as benzyloxy, phenethoxy, 3-phenylpropoxy, 1-naphthylmethoxy, 2-(1-naphtyl)ethoxy and the like.

The term “C₁₋₆-alkylsulfonyl” as used herein refers to a monovalent substituent comprising a C₁₋₆-alkyl group linked through a sulfonyl group such as e.g. methylsulfonyl, ethylsulfonyl, n-propylsulfonyl, isopropylsulfonyl, n-butylsulfonyl, sec-butylsulfonyl, isobutylsulfonyl, tert-butylsulfonyl, n-pentylsulfonyl, 2-methylbutylsulfonyl, 3-methylbutylsulfonyl, n-hexylsulfonyl, 4-methylpentylsulfonyl, neopentylsulfonyl, n-hexylsulfonyl and 2,2-dimethylpropylsulfonyl.

The term “C₁₋₆-monoalkylaminosulfonyl” as used herein refers to a monovalent substituent comprising a C₁₋₆-monoalkylamino group linked through a sulfonyl group such as e.g. methylaminosulfonyl, ethylaminosulfonyl, n-propylaminosulfonyl, isopropylaminosulfonyl, n-butylaminosulfonyl, sec-butylaminosulfonyl, isobutylaminosulfonyl, tert-butylaminosulfonyl, n-pentylaminosulfonyl, 2-methylbutylaminosulfonyl, 3-methylbutylaminosulfonyl, n-hexylaminosulfonyl, 4-methylpentylaminosulfonyl, neopentylaminosulfonyl, n-hexylaminosulfonyl and 2,2-dimethylpropylaminosulfonyl.

The term “C₁₋₆-dialkylaminosulfonyl” as used herein refers to a monovalent substituent comprising a C₁₋₆-dialkylamino group linked through a sulfonyl group such as dimethylaminosulfonyl, N-ethyl-N-methylaminosulfonyl, diethylaminosulfonyl, dipropylaminosulfonyl, N-(n-butyl)-N-methylaminosulfonyl, di(n-pentyl)aminosulfonyl, and the like.

The term “C₁₋₆-alkylsulfinyl” as used herein refers to a monovalent substituent comprising a straight or branched C₁6-alkyl group linked through a sulfinyl group (—S(═O)—); such as e.g. methylsulfinyl, ethylsulfinyl, isopropylsulfinyl, butylsulfinyl, pentylsulfinyl, and the like.

The term “C₁₋₆-alkylcarbonylamino” as used herein refers to an amino group wherein one of the hydrogen atoms is substituted with an acyl group, such as e.g. acetamido, propionamido, isopropylcarbonylamino, and the like.

The term “(C₃₋₆-cycloalkyl)C₁₋₆-alkyl” as used herein, alone or in combination, refers to a straight or branched, saturated hydrocarbon chain having 1 to 6 carbon atoms and being monosubstituted with a C₃₋₆-cycloalkyl group, the cycloalkyl group optionally being mono- or polysubstituted with C₁₋₆-alkyl, halogen, hydroxy or C₁₋₆-alkoxy; such as e.g. cyclopropylmethyl, (1-methylcyclopropyl)methyl, 1-(cyclopropyl)ethyl, cyclopentylmethyl, cyclohexylmethyl, and the like.

The term “C₁₋₆-alkylthio” or “C₁₋₆-alkylsulfanyl” as used herein, alone or in combination, refers to a straight or branched monovalent substituent comprising a lower alkyl group linked through a divalent sulfur atom having its free valence bond from the sulfur atom and having 1 to 6 carbon atoms e.g. methylsulfanyl, ethylsulfanyl, propylsulfanyl, butylsulfanyl, pentylsulfanyl.

The term “arylthio” or “arylsulfanyl” as used herein, alone or in combination, refers to an aryl group linked through a divalent sulfur atom having its free valence bond from the sulfur atom, the aryl group optionally being mono- or polysubstituted with C₁₋₆-alkyl, halogen, hydroxy or C₁₋₆-alkoxy; e.g. phenylsulfanyl, (4-methylfenyl)sulfanyl, (2-chlorophenyl)sulfanyl, and the like.

The term “arylsulfinyl” as used herein refers to an aryl group linked through a sulfinyl group (—S(═O)—), the aryl group optionally being mono- or polysubstituted with C₁-alkyl, halogen, hydroxy or C₁₋₆-alkoxy; such as e.g. phenylsulfinyl, (4-chlorophenyl)sulfinyl, and the like.

The term “arylsulfonyl” as used herein refers to an aryl group linked through a sulfonyl group, the aryl group optionally being mono- or polysubstituted with C₁₋₆-alkyl, halogen, hydroxy or C₁₋₆-alkoxy; such as e.g. phenylsulfonyl, tosyl, and the like.

The term “C₁₋₆-monoalkylaminocarbonyl” as used herein refers to a monovalent substituent comprising a C₁₋₆-monoalkylamino group linked through a carbonyl group such as e.g. methylaminocarbonyl, ethylaminocarbonyl, n-propylaminocarbonyl, isopropylaminocarbonyl, n-butylaminocarbonyl, sec-butylaminocarbonyl, isobutylaminocarbonyl, tert-butylaminocarbonyl, n-pentylaminocarbonyl, 2-methylbutylaminocarbonyl, 3-methylbutylamino-carbonyl, n-hexylaminocarbonyl, 4-methylpentylaminocarbonyl, neopentylaminocarbonyl, n-hexylaminocarbonyl and 2-2-dimethylpropylaminocarbonyl.

The term “C₁₋₆-dialkylaminocarbonyl” as used herein refers to a monovalent substituent comprising a C₁₋₆-dialkylamino group linked through a carbonyl group such as dimethylaminocarbonyl, N-ethyl-N-methylaminocarbonyl, diethylaminocarbonyl, dipropylaminocarbonyl, N-(n-butyl)-N-methylaminocarbonyl, di(n-pentyl)aminocarbonyl, and the like.

The term “C₁₋₆-monoalkylaminocarbonylamino” as used herein refers to an amino group wherein one of the hydrogen atoms is substituted with a C₁₋₆-monoalkylaminocarbonyl group, e.g. methylaminocarbonylamino, ethylaminocarbonylamino, n-propylaminocarbonylamino, isopropylaminocarbonylamino, n-butylaminocarbonylamino, sec-butylaminocarbonylamino, isobutylaminocarbonylamino, tert-butylaminocarbonylamino, and 2-methylbutylaminocarbonylamino.

The term “C₁₋₆-dialkylaminocarbonylamino” as used herein refers to an amino group wherein one of the hydrogen atoms is substituted with a C₁₋₆-dialkylaminocarbonyl group, such as dimethylaminocarbonylamino, N-ethyl-N-methylaminocarbonylamino, diethylaminocarbonylamino, dipropylaminocarbonylamino, N-(n-butyl)-N-methylaminocarbonylamino, di(n-pentyl)aminocarbonylamino, and the like.

The term “5-membered heterocyclic system” as used herein refers to: a monocyclic unsaturated or saturated system containing one, two or three hetero atoms selected from nitrogen, oxygen and sulfur and having 5 members, e.g. pyrrole, furan, thiophene, pyrroline, dihydrofuran, dihydrothiophene, imidazole, imidazoline, pyrazole, pyrazoline, oxazole, thiazole, isoxazole, isothiazole, 1,2,3-oxadiazole, furazan, 1,2,3-triazole, 1,2,3-thiadiazole or 2,1,3-thiadiazole.

The term “5- or 6-membered nitrogen, oxygen or sulfur containing ring” as used herein refers to a monovalent substituent comprising a monocyclic unsaturated or saturated system containing one or more nitrogen, oxygen or sulfur atoms and having 5 or 6 members, e.g. pyrrolidinyl, pyrrolinyl, imidazolidinyl, pyrazolidinyl, pyrazolinyl, piperidyl, piperazinyl, pyrrolyl, 2H-pyrrolyl, imidazolyl, pyrazolyl, triazolyl, pyridyl, pyrazinyl, pyrimidinyl, pyridazinyl, morpholino, thiomorpholino, isothiazolyl, isoxazolyl, oxazolyl, oxadiazolyl, thiadiazolyl, 1,3-dioxolanyl, and 1,4-dioxolanyl.

The term “base” as used herein refers to inorganic and organic bases, which can be used to make a certain transformation taking place. Useful bases are: Hydroxides as e.g. sodium, lithium, magnesium, calcium, barium, potassium or cesium hydroxide. Carbonates as e.g. sodium, lithium, magnesium, calcium, barium, potassium or cesium carbonate. Hydrogen carbonates as e.g. sodium, lithium, magnesium, calcium, barium, potassium or cesium hydrogen carbonate. Alcoholates of sodium, lithium, magnesium, calcium, barium, potassium or cesium. Alcoholates of t-butanol, methanol, ethanol, 1-propanol, 2-propanol. Tertiary amines as e.g. dimethylaminopyridine, triethylamine, diisopropylethylamine (DIPEA), pyridine, 1,5-diazabicyclo[4.3.0]non-5-ene (DBN), 1,4-diazabicyclo[2.2.2]octane (DABCO, TED), 1,8-diazabicyclo[5.4.0]undec-7-ene (DBU). Phosphates as e.g. sodium, lithium, magnesium, calcium, barium, potassium or cesium phosphate. Sulfates as e.g. sodium, lithium, magnesium, calcium, barium, potassium or cesium sulfate. Secondary amine bases as e.g. sodium, lithium, magnesium, calcium, barium, potassium or cesium bis(isopropyl)-amide and bis(cyclohexyl)amides and e.g. sodium, magnesium, calcium, barium, potassium or cesium bis(trimetylsilyl)amide. Hydrides as e.g. sodium hydride and potassium hydride. Carboxylic acid salts as e.g. sodium, lithium, magnesium, calcium, barium, potassium or cesium formate, acetate, propionate.

In one embodiment of the invention the bases are selected from the following inorganic bases: hydroxides, carbonates or hydrogen carbonates of sodium, lithium, magnesium, calcium, barium, potassium or cesium.

In another embodiment of the invention the bases are selected from the following inorganic bases: hydroxides and carbonates of sodium, lithium, magnesium, calcium, barium, potassium or cesium.

In yet another embodiment of the invention the bases are selected from sodium hydroxide, potassium carbonate, cesium carbonate, potassium hydroxide.

The term “solvent 1” as used herein refers to all solvents and combinations of solvents (e.g. mixtures of organic solvents or mixtures of one or more organic solvents and water, which can be used to make a certain transformation taking place.

In one embodiment of the invention “solvent 1” is selected from: water, organic solvents such as e.g. hydrocarbons, ethers ketones, chlorinated hydrocarbons, esters and polar solvents.

In another embodiment of the invention “solvent 1” is selected from: water, organic solvents such as hydrocarbons as e.g. toluene, xylene, hexane, heptane, ethers as e.g. t-butyl-methyl ether, tetrahydrofuran and diethyl ether, chlorinated solvents such as e.g. dichloromethane, dichloroethane, ketones such as e.g. acetone, methylisopropylketone and alkyl esters such as e.g. ethyl acetate, t-butyl acetate and isopropyl acetate and polar solvents such as e.g. N,N-dimethylformamide, N-methyl-2-pyrrolidinone, sulfolane, dimethylsulfoxide, 1,3-dimethyl-3,4,5,6-tetrahydroxy-2(1H)-pyrimidinone and acetonitrile.

In another embodiment of the invention “solvent 1” is selected from diethyl ether, acetone, toluene, t-butyl-methyl ether.

In yet another embodiment of the invention “solvent 1” is a two phase system of water and an organic solvent selected from diethyl ether, toluene or t-butyl-methyl ether or a one phase system of water and acetone or a one phase system of acetone, cyclohexanone, tetrahydrofuran, toluene, acetonitrile, N-methyl-2-pyrrolidone, N,N-dimethylformamide, dimethylsulfoxide, methanol, ethanol, 1-propanol, 2-propanol, 1-butanol, tert-butanol or pyridine.

The term “solvent 2” as used herein refers to all solvents and combinations of solvents (e.g. mixtures of organic solvents or mixtures of one or more organic solvents and water, which can be used to make a certain transformation taking place.

In one embodiment of the invention “solvent 2” is selected from: water and organic solvents such as hydrocarbons, ethers, ketones, esters, halogenated hydrocarbons, alcohols and polar solvents.

In another embodiment of the invention “solvent 2” is selected from: water, organic solvents such as hydrocarbons e.g. toluene, xylene, hexane, heptane; ethers e.g. t-butyl-methyl ether, tetrahydrofuran or diethyl ether; chlorinated solvents such as e.g. dichloromethane, dichloroethane; ketones such as e.g. acetone, methylisopropylketone; alkyl esters such as e.g. ethyl acetate, t-butyl acetate and isopropyl acetate; alcohols such as e.g. methanol, ethanol, 1-butanol, t-butanol, 1-propanol, 2-propanol or polar solvents such as e.g. N,N-dimethylformamide, N-methyl-2-pyrrolidinone, sulfolane, dimethylsulfoxide, 1,3-dimethyl-3,4,5,6-tetrahydroxy-2(1H)-pyrimidinone (DMPU) or acetonitrile.

In yet another embodiment of the invention “solvent 2” is selected from N,N-dimethylformamide, toluene, xylene,1-butanol, N-methyl-2-pyrrolidinone, sulfolane, dimethylsulfoxide, DMPU, water.

The term “metal catalyst” as used herein refers to all metal catalysts, which are capable of making the transformation taking place at lower temperatures or similar mild conditions.

In one embodiment of the invention the “metal catalyst” is selected from: copper or a copper (I) or copper (II) salt or palladium catalysts.

In another embodiment of the invention the “metal catalyst” is selected from: copper bronze, copper oxide, copper chloride, copper bromide or copper iodide, copper fluoride, copper acetate, copper acetylacetonate, copper butyrate, copper carbonate, copper cyclohexanebutyrate, copper diiron tetraoxide, copper gluconate, copper formate, copper hexaflouroacetylacetonate, copper methoxide, copper naphtenate, copper oxalate, copper perchlorate, copper phenylacetylide, copper phthalocyanide, copper selenide, copper sulfate, copper sulfide, copper tartrate, copper tetrafluoroborate, copper thiocyanate, copper triflouroacetylacetonate, copper triflouromethansulfonate, copper tungstate or a palladium catalyst such as Pd(PPh₃)₄, Pd(dba)₂/2(o-tolyl)₃P, PdCl₂(DPPF), PdCl₂(Ph₂P[CH₂]PPh₂) wherein Ph means phenyl and dba means dibenzanthrazene and copper and palladium catalysts described in catalog no 18 1999-2001 from Strem.

In yet another embodiment of the invention the “metal catalyst” is selected from copper bronze, copper oxide, copper chloride, copper bromide, copper iodide.

More particularly the present compounds of formula (I) are prepared by

a) reacting a compound of formula (II)

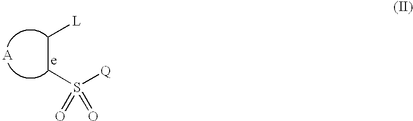

wherein A is as defined above, L is a leaving group selected from alkoxy, alkylthio, alkylsulfinyl, alkylsulfonyl, nitro or halogen and Q is halogen, with a compound of formula (III),

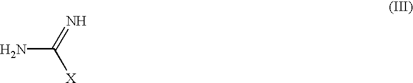

wherein X is NR²R³, wherein R² and R³ are defined above, or a suitable salt thereof, in the presence of a suitable base in solvent 1, to form a compound of formula (IV)

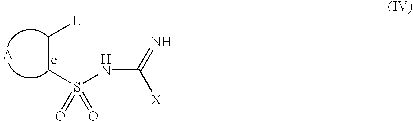

wherein A, L and X are as defined above, and thereupon cyclization of a compound of formula (IV), optionally by treatment with a metal catalyst in solvent 2, optionally in the presence of a base to form a compound of formula (I); or

b) reacting a compound of formula (II)

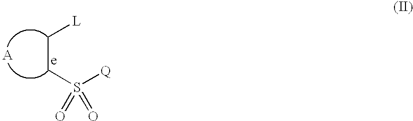

wherein A is as defined above, L is a leaving group selected from alkoxy, alkylthio, alkylsulfinyl, alkylsulfonyl, nitro or halogen and Q is halogen, with a compound of formula (III),

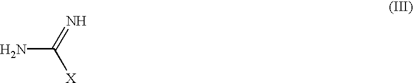

wherein X is SR¹, S(═O)R¹ or S(═O)₂R¹, wherein R¹ is defined above, or a suitable salt thereof, in the presence of a suitable base in solvent 1, to form a compound of formula (IV)

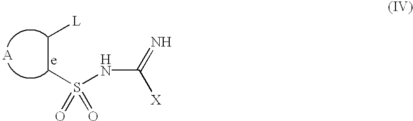

wherein A, L and X are as defined above, and thereupon cyclization of a compound of formula (IV), optionally by treatment with a metal catalyst in solvent 2, optionally in the presence of a base, to form a compound of formula (I); or

c) reacting a compound of formula (II)

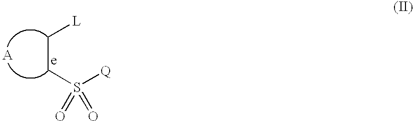

wherein A is as defined above, L is a leaving group selected from alkoxy, alkylthio, alkylsulfinyl, alkylsulfonyl, nitro or halogen and Q is halogen, with a compound of formula (III),

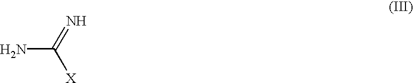

wherein X is OR¹, wherein R¹ is defined above, or a suitable salt thereof, in the presence of a suitable base in solvent 1, to form a compound of formula (IV)

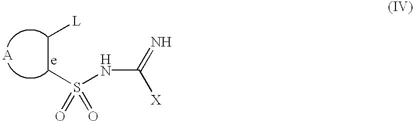

wherein A, L and X are as defined above, and thereupon cyclization of a compound of formula (IV), optionally by treatment with a metal catalyst in solvent 2, optionally in the presence of a base, to form a compound of formula (I); or

d) transforming a compound of formula (IV) into a compound of formula (IV′)

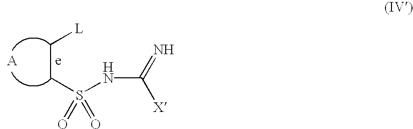

wherein A and L are as defined above and X is transformed into X′, where X′≠X (X′ is selected from the same group as X but different from the actual X), and cyclization of a compound of formula (IV′), optionally by treatment with a metal catalyst in solvent 2, optionally in the presence of a base to form a compound of formula (I); or

d′) transforming a compound of formula (IV)

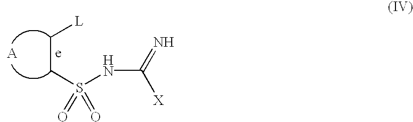

wherein A, and L are as defined above and X is SR¹, S(═O)R¹ or S(═O)₂R¹, wherein R¹ is defined above, into a compound of formula (V)

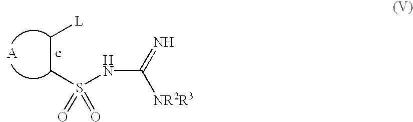

wherein A, L and R² and R³ are as defined above, and cyclization of a compound of formula (V) in solvent 2, optionally in the presence of a base, and optionally by treatment with a metal catalyst, to form a compound of formula (I); or

e) transforming a compound of formula (I), prepared as described above, by oxidation or substitution or both, to form another compound of formula (I) in analogy with known methods as described in WO97/26265.

In another embodiment of the invention cyclization of the above compound of formula (IV) in solvent 2 is carried out in the presence of a base.

In another embodiment of the invention cyclization of the above compound of formula (IV) in solvent 2 is carried out in the presence of a base and by treatment with a metal catalyst.

In another embodiment of the invention cyclization of the above compound of formula (IV) in solvent 2 is carried out in the presence of a base and without a metal catalyst.

In another embodiment of the invention cyclization of the above compound of formula (IV) in solvent 2 is not carried out in the presence of a base.

In another embodiment of the invention cyclization of the above compound of formula (IV) in solvent 2 by treatment with a metal catalyst is not carried out in the presence of a base.

In another embodiment of the invention cyclization of the above compound of formula (IV) in solvent 2 is not carried out in the presence of a base and without a metal catalyst.

Where intermediate compounds in the process of this invention are novel, such intermediates form another aspect of this invention.

Therapeutic uses of compounds selected from the group consisting of:

3-Amino-6-chloro-4H-thieno[3,2-e]-1,2,4-thiadiazine 1,1-dioxide;

7-Bromo-6-chloro-3-propylaminothieno[2,3-e]-1,2,4-thiadiazine 1,1-dioxide;

7-Bromo-3-(sec-butylamino)-6-chloro-4H-thieno[2,3-e]-1,2,4-thiadiazine 1,1-dioxide;

7-Bromo-6-chloro-3-cyclobutylamino-4H-thieno[2,3-e]-1,2,4-thiadiazine 1,1-dioxide;

6-Chloro-3-methylsulfanyl-4H-thieno[2,3-e]-1,2,4-thiadiazine 1,1-dioxide; or

6-Chloro-3-methylsulfinyl-4H-thieno[2,3-e]-1,2,4-thiadiazine 1,1-dioxide

obtained by a process of the present invention include treatment of and/or prevention of dyslipidemia, Type I diabetes, NIDDM, hypertriglyceridemia, syndrome X, insulin resistance, impaired glucose tolerance, obesity, diabetic dyslipidemia, hyperlipidemia and hypertension.

More particular, the compounds are useful in the treatment of Type I and Type II diabetes. The compounds of the invention may also be useful for the treatment of eating disorders such as anorexia or bulimia by virtue of their appetite regulating properties.

Other selected compounds are:

6-Bromo-3-methylsulfanyl-4H-thieno[2,3-e]-1,2,4-thiadiazine 1,1-dioxide;

6-Bromo-3-methylsulfinyl-4H-thieno[2,3-e]-1,2,4-thiadiazine 1,1-dioxide;

3-Amino-6-bromo-4H-thieno[3,2-e]-1,2,4-thiadiazine 1,1-dioxide;

6-Chloro-3-ethylamino-4H-thieno[2,3-e]-1,2,4-thiadiazine 1,1-dioxide;

6-Chloro-3-propylamino-4H-thieno[2,3-e]-1,2,4-thiadiazine 1,1-dioxide;

3-Isopropylamino-6-methyl-4H-thieno[3,2-e]-1,2,4-thiadiazine 1,1-dioxide;

6-Methyl-3-propylamino-4H-thieno[3,2-e]-1,2,4-thiadiazine 1,1-dioxide; or

3-sec-Butylamino-6-methyl-4H-thieno[3,2-e]-1,2,4-thiadiazine 1,1-dioxide.

Accordingly, the present invention also provides a pharmaceutical composition for treatment or prophylaxis of the disorder comprising one of the above mentioned compounds obtained using the process of the present invention and a pharmaceutically acceptable carrier, the use of one of the above mentioned compounds obtained using the process of the present invention to manufacture a medicament in solid or liquid form for the treatment or prophylaxis of the disorders and a method of treating the disorders which comprises administering an effective or prophylactic amount of one of the above mentioned compounds obtained using the process of the present invention to a person suffering from one or more of the disorder's.

The present invention is illustrated by the following examples.

The abbreviation MTBE was used for methyl tert-butyl ether.

In the examples the following HPLC method was used:

HPLC Method

Gradient HPLC Assay:

Reagents:

Acetonitrile

Trifluoroacetic acid

Millipore filtered water

HPLC Conditions:

| | |
|---|---|
| column: | 250 × 4.0 mm, 5 mm C-18 YMC-Silica 120 Å |
| | prepared at Novo Nordisk A/S |
| Flow: | 1.0 ml/min |
| Oven temp.: | 35° C. |
| Detector wavelength: | 250 nm. |
| Run time: | 40 min. |
| Gradient program: | |

| Time | Pump A | Pump B | Flow |
|---|---|---|---|
| Min. | Acetonitrile 0.1% TFA | Water 0.1% TFA | ml/min |
| 0.0 | 20 | 80 | 1.00 |
| 25.0 | 80 | 20 | 1.00 |
| 30.0 | 80 | 20 | 1.00 |
| 35.0 | 20 | 80 | 1.00 |
| 45.0 | 20 | 80 | 1.00 |

Preparation of Solutions:

Acetonitrile Containing 0.1% TFA:

Water Containing 0,1% TFA:

Isocratic HPLC Method

Reagents:

Acetonitrile,

Triethylamine

Millipore filtered water

HPLC conditions:

| | |
|---|---|
| Column: | 250 × 4.0 mm, 5 mm C-18 YMC-Silica 120 Å |
| Flow: | 1.0 ml/min |
| Detector wavelength: | 250 nm. |
| Run time: | 30 min. |

Preparation of HPLC Eluent

50% acetonitrile at pH 3:

Triethylamine (3.5 ml) was added to water (950 ml). The pH was adjusted to pH 3 with 10% phosphoric acid followed by addition of water to totally 1 l. Acetonitrile (1 l) was added and the mixture filtered (0.45 μm).

The starting materials are either known compounds or compounds, which may be prepared in analogy with the preparation of known compounds or in analogy with known methods.

### EXAMPLE 1

### 7-Bromo-6-chloro-3-methylsulfanyl-4H-thieno[2,3-e]-1,2,4-thiadiazine 1,1-dioxide

### a) N-(2,5-Dichloro-4-bromo-3-thienylsulfonyl)-S-methylisothiourea

4-Bromo-2,5-dichloro-thiophene-3-sulfonyl chloride (5.95 g, 18.0 mmol) was gradually added to a stirred mixture of S-methylisothiourea sulfate (5.2 g, 18.6 mmol) in 1N sodium hydroxide (38 ml) and ethyl ether (90 ml). The mixture was stirred at room temperature for 20 h and the ethyl ether was evaporated in vacuo to give the product as an oily residue, which solidified by stirring the heterogeneous mixture. The precipitate was isolated by filtration and recrystallised from ethanol/water to give 4.95 g (72%) of the title compound; mp 113-115° C.; ¹H-NMR (DMSO-d₆): δ2.37(s, 3H), 8.28(br s, 1H), 8.97(br s, 1H); ¹³C-NMR (DMSO-d₆): δ14.8, 110.6, 125.1, 130.2, 136.8, 171.4.

### b) 7-Bromo-6-chloro-3-methylsulfanyl-4H-thieno[2,3-e]-1,2,4-thiadiazine 1,1-dioxide

Potassium carbonate (0.69 g, 5.0 mmol) was added to a solution of N-(2,5-dichloro-4-bromo-3-thienylsulfonyl)-S-methylisothiourea (1.92 g, 5.0 mmol) in dry N,N-dimethylformamide (10 ml) and the mixture was stirred at 120° C. under nitrogen for 2 h and then concentrated to dryness in vacuo. To the residue was added water (5 ml) and 4M hydrochloric acid to pH<2, and the resulting precipitate was isolated by filtration and washed with water to give 1.38 g (79%) of the title compound; ¹H-NMR (CH₃OD): δ2.59 (s, 3H); LC-MS: m/z 347/349/351 (M+1)⁺.

### EXAMPLE 2

### 7-Bromo-6-chloro-3-methylsulfinyl-4H-thieno[2,3-e]-1,2,4-thiadiazine 1,1-dioxide

Hydrogen peroxide (35%, 2 ml) was added to a suspension of 7-bromo-6-chloro-3-methylsulfanyl-4H-thieno[2,3-e]-1,2,4-thiadiazine 1,1-dioxide (695 mg, 2.0 mmol) in acetic acid (50 ml). The mixture was stirred at room temperature for 20 h, and the white solid was isolated by filtration, washed with water and dried to give 460 mg (63%) of the title compound; ¹H-NMR (DMSO-d₆): δ2.85 (s, 3H).

### EXAMPLE 3

### 7-Bromo-6-chloro-3-propylaminothieno[2,3-e]-1,2,4-thiadiazine 1,1-dioxide

A solution of 7-bromo-6-chloro-3-methylsulfinyl-4H-thieno[2,3-e]-1,2,4-thiadiazine 1,1-dioxide (100 mg, 0.275 mmol) in propylamine (0.5 ml) was stirred for 16 h at 65° C. in a sealed glass screw cap vessel. The cooled solution was concentrated in vacuo and the residue was treated with water (3 ml) followed by adjustment to pH<2 with 4M hydrochloric acid. The product was isolated by filtration, washed with water and ethyl ether to give 90 mg (92%) of the title compound; ¹H-NMR (DMSO-d₆): δ0.89 (t, 3H), 1.52 (sext, 2H), 3.12 (q, 2H), 7.70 (br s, 1H), 11.50 (br s, 1H).

### EXAMPLE 4

### 3-Amino-6-chloro-4H-thieno[3,2-e]-1,2,4-thiadiazine 1,1-dioxide

### a) N-(3-Bromo-5-chloro-2-thienylsulfonyl)quanidine

3-Bromo-5-chloro-thiophene-2-sulfonyl chloride (0.5 g, 1.69 mmol) was added dropwise to a stirred mixture of guanidine carbonate (0.31 g, 1.72 mmol) in 1 N sodium hydroxide (3.4 ml) and ethyl ether (9 ml). The mixture was stirred at room temperature for 18 h and then the white solid was isolated by filtration to give 435 mg (81%) of the title compound; mp 236-238° C.; ¹H-NMR (DMSO-d₆): δ6.9 (br s, 4H), 7.37 (s, 1H).

### b) 3-Amino-6-chloro-4H-thieno[3,2-e]-1,2,4-thiadiazine 1,1-dioxide

A mixture of N-(3-bromo-5-chloro-2-thienylsulfonyl)guanidine (319 mg, 1.0 mmol), potassium carbonate (140 mg, 1.0 mmol) and copper bronze (10 mg) in dry N,N-dimethylformamide was stirred at 150° C. for 90 min under nitrogen. To the cooled dark mixture was added water (10 ml) and the mixture was treated with decolorizing charcoal and filtered. The filtrate was evaporated to dryness and the residue was treated with water (10 ml) and 4M hydrochloric acid to pH<2. The gray solid was isolated by filtration and recrystallized from ethanol to give 44 mg (18%) of the title compound as white crystals; mp>361° C.; ¹H-NMR (DMSO-d₆): δ7.02 (s, 1H), 7.10 (br s, 2H), 11.18 (s, 1H).

### EXAMPLE 5

### 3-Butylamino-6-chloro-4H-thieno[3,2-e]-1,2,4-thiadiazine 1,1-dioxide

A solution of 3-amino-6-chloro-4H-thieno[3,2-e]-1,2,4-thiadiazine 1,1-dioxide (50 mg, 0.21 mmol) in butylamine (0.5 ml) was stirred for 18 h at 120° C. in a sealed glass screw cap vessel. The cooled solution was concentrated in vacuo and the residue was treated with water (3 ml) followed by adjustment to pH<2 with 4M hydrochloric acid. The product was isolated by filtration, washed with water and ethyl ether to give 35 mg (57%) of the title compound; ¹H-NMR (DMSO-d₆): δ0.89 (t, 3H), 1.2-1.6 (m, 4H), 3.19 (quart, 2H), 7.05 (s, 1H), 7.31 (br s, 1H), 11.05 (br s, 1H).

### EXAMPLE 6

### 7-Bromo-3-(sec-butylamino)-6-chloro-4H-thieno[2,3-e]-1,2,4-thiadiazine 1,1-dioxide

A solution of 7-bromo-6-chloro-3-methylsulfinyl-4H-thieno[2,3-e]-1,2,4-thiadiazine 1,1-dioxide (200 mg, 0.55 mmol) in sec-butylamine (1.0 ml) was stirred for 18 h at 65° C. in a sealed glass screw cap vessel. The cooled solution was treated with water (4 ml) followed by adjustment to pH<2 with 4M hydrochloric acid. The product was isolated by filtration and washed with water to give 123 mg (60%) of the title compound; ¹H-NMR (DMSO-d₆): δ0.88 (t, 3H), 1.14 (d, 3H), 1.4-1.6 (m, 2H), 3.67 (br m, 1H), 7.55 (br s, 1H), 11.28 (br s, 1H).s, 1H).

### EXAMPLE 7

### 7-Bromo-6-chloro-3-cyclobutylamino-4H-thieno[2,3-e]-1,2,4-thiadiazine 1,1-dioxide

A solution of 7-bromo-6-chloro-3-methylsulfinyl-4H-thieno[2,3-e]-1,2,4-thiadiazine 1,1-dioxide (200 mg, 0.55 mmol) in cyclobutylamine (0.5 ml) was stirred for 18 h at 65° C. in a sealed glass screw cap vessel. The cooled solution was treated with water (4 ml) followed by adjustment to pH<2 with 4M hydrochloric acid. The product was isolated by filtration, washed with water and recrystallized from methanol/water to give 72 mg (35%) of the title compound; mp 341-42° C. dec.; ¹H-NMR (DMSO-d₆): δ1.55-1.78 (m, 2H), 1.87-2.12 (m, 2H), 2.14-2.34 (m, 2H), 4.12 (br sext, 1H), 8.02 (br d, 1H), 11.34 (br s, 1H).

### EXAMPLE 8

### 6-Chloro-3-isopropylamino-4H-thieno[3,2-e]-1,2,4-thiadiazine 1,1-dioxide

### a) 3-Bromo-5-chlorothiophene-2-sulfonyl chloride

2-Chloro-4-bromothiophene (10 g) was added to chlorosulfonic acid (13.4 ml) at 0-5° C. under an atmosphere of nitrogen. After the addition, the reaction mixture was stirred at 0-5° C. for about 30 min and then added to another flask containing water (20 ml), methyl-tert-butylether (MTBE) (30 ml) and heptane (30 ml) under stirring at 0-5° C. The two phases were separated and the aqueous phase extracted with a mixture of heptane (25 ml) and MTBE (25 ml). The combined organic phases were dried with MgSO₄ and evaporated in vacuo to give the crude product as a purple oil (13.4 g). HPLC purity >84% (isocratic HPLC method); ¹H-NMR (CDCL₃): δ: 7.09 (s, 1H). The crude product was used without further purification.

### b) N-(3-Bromo-5-chloro-2-thienylsulfonyl)-N′-isopropylguanidine

Isopropylguanidine hydrochloride (4.65 g), 2N sodium hydroxide (50 ml) and MTBE (250 ml) were stirred until all the isopropylguanidine hydrochloride was dissolved. Crude 3-bromo-5-chlorothiophene-2-sulfonyl chloride (10 g) dissolved in MTBE (25 ml) was added dropwise during 20 min at room temperature. The reaction mixture was stirred for 2 h until no starting material could be detected. The two phases were separated and the organic phase was extracted with ethyl acetate (3×50 ml). The combined organic phases were dried with magnesium sulfate and the solvent was partly evaporated in vacuo to a volume of 40-70 ml. The formed crystals were separated by filtration to give 6.4 g of the title product. The filtrate was evaporated in vacuo to give 5 g of an oil which contained some title product; ¹H-NMR (CDCl₃): δ1.10 (d, 6H), 3.58-4.00 (m, 1H), 6.59 (br s, 1H), 7.03 (br s, 1H), 7.38 (s, 1H), 7.15-7.50 (br s 1H). C₈H₁₁N₂O₂S₂BrCl: calc. C, 26.6; H, 3.1; N, 11.65; found C, 26.85; H, 3.1; N, 11.6.

### c) 6-Chloro-3-isopropylamino-4H-thieno[3,2-e]-1,2,4-thiadiazine 1,1-dioxide

A mixture of N-(3-Bromo-5-chloro-2-thienylsulfonyl)-N′-isopropylguanidine (2 g), copper bronze (0.05 g), potassium carbonate (2 g), and a crystal of iodine was stirred in dry N,N-dimethylformamide (40 ml) in a sealed glass screw cap vessel at 125° C. for 20 h. The reaction mixture was filtered and the filter cake washed with N,N-dimethylformamide (2×10 ml). The pooled organic phases were evaporated to dryness, water (80 ml) was added and the suspension filtered. The pH of the aqueous phase was adjusted to <2 with 2N hydrochloric acid and the precipitated crude product was filtered off, washed with water (10 ml) and dried in vacuo (1.1 g, 72%).

### EXAMPLE 9

### 6-Chloro-3-isopropylamino-4H-thieno[3,2-e]-1,2,4-thiadiazine 1,1-dioxide

The same procedure as described in example 8c was used (N-(3-bromo-5-chloro-2-thienyl-sulfonyl)-N′-isopropylguanidine (0.2 g), copper bronze (10 mg), iodine (one crystal)) together with procedures where copper and iodine (Cu, I₂) were substituted with copper(I) oxide (CuO) (22.9 mg), copper(I) chloride (CuCl) (15.8 mg), copper(I) bromide (CuBr) (23 mg), copper(I) iodide (CuI) (30.5 mg) and omitted. After 1 h. the following ratios were obtained (the ratios are calculated as the ratio between product and starting material): Cu, I₂: 96% product; CuO: 96%; CuCl: 98%, CuBr: 92%; CuI: 94%; no Cu:<1%. After 3 h. the following results were obtained: Cu, I₂: 99% product; CuO: 99%; CuCl: 100%, CuBr: 100%; CuI: 100%; no Cu: 1%. After 18 h.: no Cu: 10%. The analytical results were obtained by using the described gradient HPLC assay.

### EXAMPLE 10

### 6-Chloro-3-isopropylamino-4H-thieno[3,2-e]-1,2,4-thiadiazine 1,1-dioxide

A mixture of N-(3-Bromo-5-chloro-2-thienylsulfonyl)-N′-isopropylguanidine (200 mg), copper(I) oxide (23 mg), and potassium hydroxide (160 mg) was stirred in dry N,N-dimethylformamide (5 ml) in a sealed glass screw cap vessel at 120° C. After 3 hours the amount of product formed was 94%. After 6 h the product ratio (calculated as the ratio between product and starting material) was 97%.

### EXAMPLE 11

### 6-Chloro-3-isopropylamino-4H-thieno[3,2-e]-1,2,4-thiadiazine 1,1-dioxide

N-(3-Bromo-5-chloro-2-thienylsulfonyl)-N′-isopropylguanidine (200 mg) was added to a glass screw cap vessel together with copper(I) oxide (2 mg), potassium carbonate (200 mg) and dry N,N-dimethylformamide (5 ml), sealed and heated to 120° C. After 3 h the amount of product formed was 85%. After 6 h the product ratio (calculated as the ratio between product and starting material) was 98%.

### EXAMPLE 12

### 6-Chloro-3-isopropylamino-4H-thieno[3,2-e]-1,2,4-thiadiazine 1,1-dioxide

N-(3-Bromo-5-chloro-2-thienylsulfonyl)-N′-isopropylguanidine (200 mg) was added to a glass screw cap vessel together with copper(I) oxide (23 mg), potassium carbonate (200 mg) and the following solvents: N-methyl-2-pyrolidone (NMP) (5 ml), sulfolane (5 ml), dimethylsulfoxide (DMSO) (5 ml), 1,3-dimethyl-3,4,5,6-tetrahydroxy-2(1H)-pyrimidinone (DMPU) (5 ml). After 1 h the product ratio (calculated as the ratio between product and starting material) was NMP: 84%, Sulfolane: 45%, DMSO: 78%, DMPU 67%. After 3 h the amount of product formed was NMP:≡100%, Sulfolane: 92%, DMSO:≡100%, DMPU≡100%.

### EXAMPLE 13

### 6-Chloro-3-isopropylamino-4H-thieno[3,2-e]-1,2,4-thiadiazine 1,1-dioxide

N-(3-Bromo-5-chloro-2-thienylsulfonyl)-N′-isopropylguanidine (200 mg) was added to a glass screw cap vessel together with copper(I) oxide (2 mg), potassium hydroxide (160 mg) and dry toluene (10 ml), sealed and heated to 120° C. After 6 h the product ratio (calculated as the ratio between product and starting material) was 98%.

### EXAMPLE 14

### 3-Benzylsulfanyl-6-chloro-4H-thieno[3,2-e]-1,2,4-thiadiazine 1,1-dioxide

### a) N-(3-Bromo-5-chloro-2-thienylsulfonyl)-S-benzylisothiourea

S-Benzyl-thiuronium chloride (8.1 g), sodium hydroxide (2.2 g) and acetone (100 ml) were stirred for 5 min before crude 3-bromo-5-chlorothiophene-2-sulfonyl chloride (10 g) was added dropwise during 20 min at room temperature. The reaction mixture was stirred for 24 h. The suspension was evaporated in vacuo and the crude reaction mixture partitioned between 1N hydrochloric acid (30 ml) and dichloromethane (50 ml). The two phases were separated and the aqueous phase extracted with dichloromethane (4×50 ml). The combined organic phases were dried with magnesium sulfate and evaporated in vacuo to give the crude product (12.8 g). The crude product was suspended in toluene and the crystals isolated by filtration (4.0 g); ¹H-NMR (CDCl₃) δ: 4.23 (s, 2H), 7.15-7.35 (m, 5H), 7.44 (s, 1H), 8.0-8.5 (br s, 1H), 8.8-9.4 (br s, 1H); ¹³C-NMR (CDCl₃) δ: 33.1, 114.8, 125.6, 126.5, 127.1, 134.2, 135.0, 137.1, 157.1.

### b) 3-Benzylsulfanyl-6-chloro-4H-thieno[3,2-e]-1,2,4-thiadiazine 1,1-dioxide

N-(3-Bromo-5-chloro-2-thienylsulfonyl)-S-benzylisothiourea (2 g) was added to a glass screw cap vessel together with copper (0.05 g), potassium carbonate (2 g), iodine (one crystal) and dry N,N-dimethylformamide (30 ml), sealed and heated to 125° C. for 1½ h. The reaction mixture was filtered and the filter cake washed with N,N-dimethylformamide (2×10 ml). The pooled organic phases were evaporated to dryness, water (100 ml) was added and the suspension filtered. The pH of the aqueous phase was adjusted to <2 with 2N hydrochloric acid and the precipitated crude product was filtered off, washed with water (10 ml) and dried in vacuo (65 mg); HPLC-purity (isocratic method): 96%; ¹H-NMR (CDCl₃) δ: 4.42 (s, 2H), 7.02 (s, 1H) 7.15-7.76 (m, 5H).

### EXAMPLE 15

### 6-Chloro-3-methylsulfanyl-4H-thieno[3,2-e]-1,2,4-thiadiazine 1,1-dioxide

### a) N-(3-Bromo-5-chloro-2-thienylsulfonyl)-S-methylisothiourea

A mixture of S-methylthiuronium sulfate (9.4 g), 2N sodium hydroxide (34 ml) and ethyl ether (180 ml) was stirred for 5 min. Crude 3-bromo-5-chlorothiophene-2-sulfonyl chloride (10 g) dissolved in ethyl ether (20 ml) was added dropwise during 30 min under vigorously stirring at room temperature. The reaction mixture was stirred for 24 h. and the two phases were separated. The organic phase was extracted with water (3×50 ml), dried with magnesium sulfate, filtered and evaporated in vacuo (10.1 g). Crystallization of the crude product from a mixture of acetone and water provided the title product (8 g); ¹H-NMR (CDCl₃): δ: 2.45 (s, 3H), 6.93 (s, 1H), 5.5-6.8 (br s, 1H), 7.4-8.5 (br s, 1H); ¹³C-NMR (CDCl₃): δ14.7, 112.0, 133.0, 134.1, 138.6, 171.3.

### b) 6-Chloro-3-methylsulfanyl-4H-thieno[3,2-e]-1,2,4-thiadiazine 1,1-dioxide

N-(3-Bromo-5-chloro-2-thienylsulfonyl)-S-methylthiourea (2 g) was added to a glass screw cap vessel together with copper (60 mg), potassium carbonate (2 g), iodine (a crystal) and dry N,N-dimethylformamide (40 ml), sealed and heated to 120° C. for 3 h. The reaction mixture was filtered and the filter cake washed with N,N-dimethylformamide (2×10 ml). The pooled organic phases were evaporated to dryness, water (100 ml) was added and the pH was adjusted to <2 with 2N hydrochloric acid. The suspension was filtered and the crude product washed with dichloromethane (10 ml) and dried in vacuo (0.2 g); ¹H-NMR (DMSO-d₆): δ2.50 (s, 3H), 7.00 (s, 1H) 12.8-13.5 (br s, 1H); ¹H-NMR (MeOD-d₄): δ2.59 (s, 3H), 6.84 (s, 1H); ¹³C-NMR (NMR (MeOD-d₄): δ14.3, 118.5, 126.3, 136.2, 139.8, 160.4.

### EXAMPLE 16

### 6-Bromo-3-isopropylamino-4H-thieno[3,2-e]-1,2,4-thiadiazine 1,1-dioxide

### a) N-(3,5-Dibromo-2-thienylsulfonyl)-N′-isopropylquanidine

A solution of 3,5-dibromothiophene-2-sulfonyl chloride (5.0 g, 14.7 mmol) in toluene (30 ml) was added dropwise to a stirring mixture of isopropylguanidine p-toluene sulfonate (4.0 g, 14.7 mmol) in 35 ml of 1 N sodium hydroxide and 30 ml of toluene. The mixture was stirred at room temperature for 30 min, the phases were separated and the aqueous phase extracted with dichloromethane. The combined organic phases were dried, MgSO₄ and evaporated to dryness to give a yellow oil. The oil was crystallized by trituration with ethyl acetate/heptane to give 1.87 g (32%) of the title compound; mp 146-148° C.

### b) 6-Bromo-3-isopropylamino-4H-thieno[3,2-e]-1,2,4-thiadiazine 1,1-dioxide

A mixture of N-(3,5-dibromo-2-thienylsulfonyl)-N-isopropylguanidine(1.0 g, 2.5 mmol), potassium carbonate (0.44 g, 3.2 mmol) and copper bronze (40 mg) in dry N,N-dimethylformamide (10 ml) was stirred at 150° C. for 1 h under nitrogen. To the cooled mixture was added 20 ml of water and the mixture was treated with decolorizing charcoal and filtered. The filtrate was evaporated to dryness and the residue was treated with 5 ml of water and 4 M hydrochloric acid to pH<2. The solid was isolated by filtration, washed with water and dried to give 0.48 g (59%) of the title compound; mp 279-281° C.; ¹H-NMR (DMSO-d₆): δ d 1.16 (d, 6H), 3.86 (m, 1H), 7.14 (s, 1H), 7.18 (br, 1H), 10.74 (s, 1H).

### EXAMPLE 17

### 6-Chloro-3-methylsulfanyl-4H-thieno[2,3-e]-1,2,4-thiadiazine 1,1-dioxide

### a) N-(2,5-Dichloro-3-thienylsulfonyl)-S-methylisothiourea

2,5-Dichlorothiophene-3-sulfonyl chloride (5.0 g, 19.8 mmol) was gradually added to a stirred mixture of S-methylisothiourea sulfate (5.5 g, 19.8 mmol) in 40 ml of 1N sodium hydroxide and 50 ml of ether. The mixture was stirred at room temperature for 20 h, the phases separated and the ether phase was evaporated in vacuo to give an oily product, which slowly solidified. ¹H-NMR (DMSO-d₆): δ2.36 (s, 3H), 7.40 (s, 1H), 8.20 (br s, 1H), 8.95 (br s, 1H). The crude product was used in the next step without further purification.

### b) 5-Chloro-3-methylsulfanyl-4H-thieno[2,3-e]-1,2,4-thiadiazine 1,1-dioxide

A mixture of N-(2,5-dichloro-3-thienylsulfonyl)-S-methylisothiourea (5.50 g, 18.0 mmol), potassium carbonate (2.48 g, 18.0 mmol) and copper bronze (100 mg) in dry N,N-dimethylformamide (30 ml) was stirred at 150° C. for 2 h under nitrogen. To the cooled mixture was added 50 ml of water and the mixture was treated with decolorizing charcoal and filtered. The filtrate was evaporated to dryness and the residue was treated with 25 ml of water and 4 M hydrochloric acid to pH<2. The solid was isolated by filtration, washed with water and ether to give 2.18 g (45%) of the title compound; mp 324-326° C.; ¹H-NMR (DMSO-d₆): δ d 2.50 (s, 3H), 7.44 (s, 1H).

### EXAMPLE 18

### 6-Chloro-3-methylsulfinyl-4H-thieno[2,3-e]-1,2,4-thiadiazine 1,1-dioxide

Hydrogen peroxide (35%, 1.2 ml) was added to a suspension of 6-chloro-3-methylsulfanyl-4H-thieno[2,3-e]-1,2,4-thiadiazine 1,1-dioxide (0.35 g, 1.3 mmol) in acetic acid (25 ml), and the mixture was stirred at room temperature for 24 h. Water (75 ml) was added to the resulting solution which was extracted with dichloromethane (50 ml). The organic phase was dried with magnesium sulfate and evaporated to give 170 mg (46%) of the title compound as white crystals; ¹H-NMR (DMSO-d₆): δ2.93 (s, 3H), 7.46 (s, 1H).

### EXAMPLE 19

### 6-Chloro-3-isopropylamino-4H-thieno[3,2-e]-1,2,4-thiadiazine 1,1-dioxide

N-(3-Bromo-5-chloro-2-thienylsulfonyl)-N-isopropylguanidine (10 g, 27.7 mmol) was added to xylene (230 ml) under an atmosphere of nitrogen. Copper(I) oxide (74 mg, 0.52 mmol), cesium carbonate (13.5 g, 41.5 mmol) and water (2.3 ml) was added and the reaction mixture was heated to 120-125° C. for 12-14 h. After cooling to room temperature methanol (60 ml) was added and the reaction mixture was filtered. The crystal mass was washed with methanol (2×10 ml) and water (230 ml) was added to the solvent mixture. The two phases were separated and the xylene phase was washed with a mixture of water and methanol (2:1; 2×35 ml). Methanol was evaporated in vacuo from the aqueous/methanol phase and 1 M hydrochloric acid (about 35 ml) was added to pH 1-2 under vigorous stirring. The precipitated crude product was isolated by filtration, washed with 1M hydrochloric acid (2×5 ml) and recrystallized from acetic acid to give 4.0 g (52%) of the title compound.

### EXAMPLE 20

### 6-Chloro-3-isopropylamino-4H-thieno[3,2-e]-1,2,4-thiadiazine 1,1-dioxide

N-(3-Bromo-5-chloro-2-thienylsulfonyl)-N′-isopropylguanidine (100 mg) was added to a glass screw cap vessel together with copper(II) chloride (2.2 mg), cesium carbonate (136 mg), xylene (5 ml) and water (20 μl). The vessel was sealed and heated to 115° C. After 21 h the amount of product formed was 81% (the data are found as described in example 9).

### EXAMPLE 21

### 6-Chloro-3-isopropylamino-4H-thieno[3,2-e]-1,2,4-thiadiazine 1,1-dioxide

N-(3-Bromo-5-chloro-2-thienylsulfonyl)-N′-isopropylguanidine (100 mg) was added to a glass screw cap vessel together with copper(I) oxide (2.3 mg), cesium carbonate (136 mg), n-butanol (5 ml) and water (20 μl). The vessel was sealed and heated to 115° C. After 21 h a product ratio (calculated as the ratio between product and starting material) of about 100% was seen.

### EXAMPLE 22

### 6-Bromo-3-methylsulfanyl-4H-thieno[2,3-e]-1,2,4-thiadiazine 1,1-dioxide

### a) N-(2,5-Dibromo-3-thienylsulfonyl)-S-methylisothiourea

2,5-Dibromothiophene-3-sulfonyl chloride (5.1 g, 15.0 mmol) was added to a stirred mixture of S-methylisothiourea sulfate (4.3 g, 15.4 mmol) in 1N sodium hydroxide (45 ml) and ethyl ether (75 ml). The mixture was stirred at room temperature for 1 h and the ethyl ether was evaporated in vacuo. The resulting oil/water mixture was acidified with 4M hydrochloric acid (5 ml) and the oily product was isolated by decantation and finally triturated with water (50 ml) to give the product as a solid. The crude product was washed with water and dried to give 3.42 g (58%) of the title compound; ¹H-NMR (DMSO-d₆): δ2.37(s, 3H), 7.48(s, 1H), 8.14(br s, 1H), 8.95(br s, 1H); LC-MS: m/z 393/395/397 (M+1)⁺.

### b) 6-Bromo-3-methylsulfanyl-4H-thieno[2,3-e]-1,2,4-thiadiazine 1,1-dioxide

Potassium carbonate (1.11 g, 8.0 mmol) and copper bronze (100 mg) was added to a solution of N-(2,5-dibromo-3-thienylsulfonyl)-S-methylisothiourea (3.15 g, 8.0 mmol) in dry N,N-dimethylformamide (15 ml) and the mixture was stirred at 100° C. under nitrogen for 90 min. The cooled mixture was filtered and the resulting filtrate was concentrated to dryness. The residue was taken up in water (25 ml), treated with decolorizing charcoal and filtered. The filtrate was acidified with 1M hydrochloric acid to pH<2 and the resulting precipitate was isolated by filtration, washed with water and dried to give 0.97 g (39%) of the title compound; ¹H-NMR (DMSO-d₆): δ2.51(s, 3H), 7.52(s, 1H) (the NH peak was hidden under the water peak).

### EXAMPLE 23

### 6-Bromo-3-methylsulfinyl-4H-thieno[2,3-e]-1,2,4-thiadiazine 1,1-dioxide

Hydrogen peroxide (35%, 2.6 ml) was added to a stirred suspension of 6-bromo-3-methylsulfanyl-4H-thieno[2,3-e]-1,2,4-thiadiazine 1,1-dioxide (0.88 g, 2.8 mmol) in acetic acid (50 ml). The mixture was stirred at room temperature for 22 h, and the resulting yellow solution was poured into water (100 ml). The solution was extracted with dichloromethane (3×50 ml) and the organic phase was dried over sodium sulfate and evaporated to dryness. The crude product was triturated with a small amount of ethyl ether to give 0.55 g (60%) of the title compound; ¹H-NMR (DMSO-d₆): δ2.94(s, 3H), 7.56(s, 1H), (the NH-proton was hidden under a broad water peak at ca. 7.5 ppm.).

### EXAMPLE 24

### 3-Amino-6-bromo-4H-thieno[3,2-e]-1,2,4-thiadiazine 1,1-dioxide

### a) N-(3,5-Dibromo-2-thienylsulfonyl)quanidine

3,5-Dibromothiophene-2-sulfonyl chloride (7.0 g, 20.5 mmol) was added in portions to a stirred mixture of guanidine carbonate (3.6 g, 20.7 mmol) in 2N sodium hydroxide (20 ml, 40 mmol) and ethyl ether (100 ml). The mixture was stirred at room temperature for 20 h and the two phases were separated. The aqueous layer was washed with ethyl ether (2×50 ml), diluted with 100 ml of water and stirred until the product solidified. The precipitate was isolated by filtration, washed with water and dried to give 4.8 g (64%) of the title compound; ¹H-NMR (DMSO-d₆): δ6.9 (br s, 4H), 7.41 (s, 1H); MS: m/z 361/363/365 (M⁺); (C₅H₅N₃Br₂O₂S₂) calc. C, 16.54; H, 1.39; N, 11.57; found C, 16.90; H, 1.39; N, 11.28.

### b) 3-Amino-6-bromo-4H-thieno[3,2-e]-1,2,4-thiadiazine 1,1-dioxide

A mixture of N-(3,5-dibromo-2-thienylsulfonyl)guanidine (363 mg, 1.0 mmol), potassium carbonate (140 mg, 1.0 mmol) and copper bronze (10 mg) in dry N,N-dimethylformamide (2 ml) was stirred at 150° C. for 2 h under nitrogen. Water (20 ml) was added to the cooled dark mixture followed by treatment with decolorizing charcoal. After filtration, the filtrate was evaporated to dryness and the residue was dissolved in water (3 ml) and adjusted to pH<2 with 4M hydrochloric acid. The solid, which precipitated, was isolated by filtration, washed with water and dried to give 174 mg (62%) of the crude title compound; ¹H-NMR (DMSO-d₆): δ7.09 (br s, 3H), 11.14 (br s, 1H); LC-MS: m/z 282/284 (M+1)⁺.

### EXAMPLE 25

### 6-Chloro-3-isopropylamino-4H-thieno[2,3-e]-1,2,4-thiadiazine 1,1-dioxide

### a) N-(2,5-Dichloro-3-thienylsulfonyl)-N′-isopropylquanidine

A solution of 2,5-dichlorothiophene-3-sulfonyl chloride (10.0 g, 39.8 mmol) in ethyl ether (50 ml) was added to a stirred mixture of N-isopropylguanidine tosylate (10.9 g, 39.8 mmol) in 1N sodium hydroxide (80 ml) and ethyl ether (50 ml), and the mixture was stirred at room temperature for 1 h. The two phases were separated and the organic layer was left overnight to give a precipitate which was isolated by filtration, washed with ethyl ether and dried affording 7.8 g (62%) of the title compound; ¹H-NMR (DMSO-d₆): δ1.06 (d, 6H), 3.8 (br s, 1H), 6.4-7.5 (broad peaks, 3H), 7.28 (s, 1H). The crude product was used in the next step without further purification.

### b) 6-Chloro-3-isopropylamino-4H-thieno[2,3-e]-1,2,4-thiadiazine 1,1-dioxide

A mixture of N-(2,5-dichloro-3-thienylsulfonyl)-N′-isopropylguanidine (7.0 g, 22.1 mmol), potassium carbonate (3.05 g, 22.1 mmol) and copper bronze (30 mg) in dry N,N-dimethylformamide (70 ml) was stirred at 1500 C for 3½ h under nitrogen. The cooled mixture was evaporated to dryness and the residue was partly dissolved in 1N sodium hydroxide (50 ml) by gently heating. The mixture was treated with decolorizing charcoal, filtered and the filtrate was acidified with 4M hydrochloric acid to pH<2. The resulting dark solid was isolated by filtration, washed with water and recrystallized from ethanol to give 2.45 g (39%) of the pure title compound; mp 271-272° C.; ¹H-NMR (DMSO-d₆): δ1.16 (d, 6H), 3.85 (m, 1H), 7.23 (s, 1H), 7.48 (br d, 1H), 11.12 (s, 1H); (C₈H₁₀N₃ClO₂S₂) calc. C, 34.35; H, 3.60; N, 15.02; found C, 34.54; H, 3.63; N, 14.84.

### EXAMPLE 26

### 6-Chloro-3-ethylamino-4H-thieno[2,3-e]-1,2,4-thiadiazine 1,1-dioxide

### a) N-(2,5-Dichloro-3-thienylsulfonyl)-N′-ethylguanidine

A solution of 2,5-dichlorothiophene-3-sulfonyl chloride (5.0 g, 19.9 mmol) in ethyl ether (25 ml) was added to a stirred mixture of N-ethylguanidine hydrochloride (2.46 g, 19.9 mmol) in 1N sodium hydroxide (40 ml) and ethyl ether (25 ml), and the mixture was stirred at room temperature for 1 h. The solid, which precipitated was filtered off, washed with ethyl ether and dried to give 5.03 g (84%) of the title compound; ¹H-NMR (DMSO-d₆): δ1.02 (t, 3H), 3.11 (quint, 2H), 6.6-7.5 (broad peaks, 3H), 7.28 (s, 1H). The crude product was used in the next step without further purification.

### b) 6-Chloro-3-ethylamino-4H-thieno[2,3-e]-1,2,4-thiadiazine 1,1-dioxide

A mixture of N-(2,5-dichloro-3-thienylsulfonyl)-N′-ethylguanidine (4.0 g, 13.2 mmol), potassium carbonate (1.83 g, 13.2 mmol) and copper bronze (140 mg) in dry N,N-dimethylformamide (40 ml) was stirred at 150° C. for 5 h under nitrogen. The cooled dark mixture was filtered and the filter was washed with a small portion of water. The combined filtrate was evaporated to dryness and the residue was stirred with water (100 ml) and adjusted to pH<2 with 4M hydrochloric acid. The formed precipitate was dissolved in 1 N sodium hydroxide (50 ml), treated with decolorizing charcoal, filtered and the filtrate was adjusted to pH<2 with 4M hydrochloric acid. The resulting solid was isolated by filtration, washed with water, recrystallized from ethanol and finally purified by column chromatography using dichloromethane/methanol (50:1) to give 678 mg (19%) of the title compound; mp 274-275° C.; ¹H-NMR (DMSO-d₆): δ1.11 (t, 3H), 3.20 (m, 2H), 7.23 (s, 1H), 7.55 (br t, 1H), 11.36 (br s, 1H); MS: m/z 265/267 (M⁺); (C₇H₈N₃ClO₂S₂) calc. C, 31.64; H, 3.03; N, 15.81; found C, 31.64; H, 3,20; N, 15.18.

### EXAMPLE 27

### 6-Chloro-3-propylamino-4H-thieno[2,3-e]-1,2,4-thiadiazine 1,1-dioxide

### a) N-(2,5-Dichloro-3-thienylsulfonyl)-N′-propylguanidine

A solution of 2,5-dichlorothiophene-3-sulfonyl chloride (5.0 g, 19.9 mmol) in ethyl ether (25 ml) was added to a stirred mixture of N-propylguanidine hydrochloride (2.74 g, 19.9 mmol) in 1N sodium hydroxide (40 ml) and ethyl ether (25 ml), and the mixture was stirred at room temperature for 1 h. The solid, which precipitated was filtered off, washed with ethyl ether and dried to give 3.3 g (56%) of the title compound; ¹H-NMR (DMSO-d₆): δ0.83 (t, 3H), 1.42 (sext, 2H), 3.04 (br t, 2H), 6.6-7.6 (broad peaks, 3H), 7.27 (s, 1H). The crude product was used in the next step without further purification.

### b) 6-Chloro-3-propylamino-4H-thieno[2,3-e]-1,2,4-thiadiazine 1,1-dioxide

A mixture of N-(2,5-dichloro-3-thienylsulfonyl)-N′-propylguanidine (3.23 g, 10.2 mmol), potassium carbonate (1.41 g, 10.2 mmol) and copper bronze (130 mg) in dry N,N-dimethylformamide (30 ml) was stirred at 150° C. for 4 h under nitrogen. The cooled dark mixture was filtered and the filter was washed with a small portion of water. The combined filtrate was evaporated to dryness and the residue was stirred with water (100 ml) and adjusted to pH<2 with 4M hydrochloric acid. The formed precipitate was dissolved in 1 N sodium hydroxide (50 ml) by gently heating, treated with decolorizing charcoal, filtered and the filtrate was adjusted to pH<2 with 4M hydrochloric acid. The resulting solid was isolated by filtration, washed with water, recrystallized from ethanol and finally purified by column chromatography using dichloromethane/methanol (50:1) to give 801 mg (28%) of the title compound; mp 246-247° C.; ¹H-NMR (DMSO-d₆): δ0.90 (t, 3H), 1.52 (sext, 2H), 3.12 (dt, 2H), 7.23 (s,1H), 7.57 (br t, 1H), 11.30 (br s, 1H).; MS: m/z 279/281 (M⁺); (C₈H₁₀N₃ClO₂S₂) calc. C, 34.35; H, 3.60; N, 15.02; found C, 34.34; H, 3.68; N, 14.81.

### EXAMPLE 28

### 6-Chloro-3-methoxy-4H-thieno[3,2-e]-1,2,4-thiadiazine 1,1-dioxide

### a) N-(3-Bromo-5-chloro-2-thienylsulfonyl)-O-methyl-isourea

A solution of 3-bromo-5-chlorothiophene-2-sulfonyl chloride (2.0 g, 6.76 mmol) in ethyl ether (5 ml) was added to a stirred mixture of O-methylisourea sulfate (1.0 g, 4.05 mmol) in 1N sodium hydroxide (15 ml) and ethyl ether (10 ml), and the mixture was stirred at room temperature for 4 h. A further amount of O-methylisourea sulfate (1.0 g, 4.05 mmol) in 1N sodium hydroxide (8 ml) was added and the mixture was stirred overnight. The mixture was adjusted to pH 6-7 with 4M hydrochloric acid and the two phases were separated. The aqueous phase was extracted with ethyl ether (2×25 ml) and the combined organic layers were dried over sodium sulfate and evaporated to dryness. The residue was triturated with petroleum ether and dried to give 1.21 g (54%) of the title compound; ¹H-NMR (CDCl₃): δ3.86 (s, 3H), 5.42 (br s, 1H), 6.93 (s, 1H), 7.32 (br s, 1H). The crude product was used in the next step without further purification.

### b) 6-Chloro-3-methoxy-4H-thieno[3,2-e]-1,2,4-thiadiazine 1,1-dioxide

A mixture of N-(3-bromo-5-chloro-2-thienylsulfonyl)-O-methyl-isourea (0.60 g, 1.8 mmol), potassium carbonate (0.25 g, 1.8 mmol) and a catalytic amount of copper bronze in dry N,N-dimethylformamide (4 ml) was stirred at 120° C. for 3 h under nitrogen. Water (15 ml) was added to the cooled dark mixture, which was treated with decolorizing charcoal, and filtered. The filtrate was evaporated to dryness and the residue was stirred in water (10 ml) and finally adjusted with 4M hydrochloric acid to pH<2. The resulting solid was isolated by filtration, washed with water and ethyl ether to give 150 mg (33%) of the crude title compound; ¹H-NMR (DMSO-d₆): δ3.90 (s, 3H), 6.99 (s, 1H), 12.70 (br s, 1H).

### EXAMPLE 29

### 3-Isopropylamino-6-methyl-4H-thieno[3,2-e]-1,2.4-thiadiazine 1,1-dioxide

### a) 3-Bromo-5-methylthiophene-2-sulfinic acid

A solution of diisopropylamine (10.65 ml, 76.0 mmol) in dry tetrahydrofuran (250 ml) under nitrogen was cooled to −30° C., and n-butyllithium (51 ml of a 1.33M solution in hexane) was added dropwise over a period of 10 min with magnetic stirring. Stirring was continued at −30° C. to 40° C. for 30 min, and then the mixture was cooled to −70° C. and quickly added a solution of 2-bromo-5-methylthiophene (6.44 ml, 56 mmol) in dry tetrahydrofuran (5 ml). The mixture was stirred at −75° C. for 2½ h, and then a rapid stream of dry gaseous sulfur dioxide was bubbled just below the surface of the stirred mixture at −75° C. until an aliquot quenched into water was acidic. The mixture was allowed to warm to room temperature, and then the excess sulfur dioxide and solvents were evaporated in vacuo and the residue was triturated overnight with ethyl ether (50 ml). Filtration gave 16.7 g of a mixture of lithium and diisopropyl ammonium sulfinic acid salts. The mixture was dissolved in water (50 ml) and lithium hydroxide (1.4 g, 58 mmol) was added. The resulting solution was treated with decolorizing charcoal, and filtered. The filtrate was thoroughly evaporated to dryness in vacuo (in the presence of at least one equivalent of 1-propanol to avoid foaming), and the residue was triturated with acetone to leave 8.0 g (58%) of almost pure lithium salt of the title compound; mp>400° C.; ¹H-NMR (D₂O): δ2.50 (s, 3H), 6.80 (s, 1H).

### b) 3-Bromo-5-methylthiophene-2-sulfonyl chloride

The lithium salt of 3-bromo-5-methylthiophene-2-sulfinic acid (2.47 g, 10 mmol) was dissolved in an ice cooled mixture of acetic acid (5 ml) and water (5 ml) with magnetic stirring. N-Chlorosuccinimide (1.40 g, 10 mmol) was added in small portions, and the mixture was stirred at room temperature for 35 min. The mixture was diluted with water (10 ml) and filtered to give 1.3 g (47%) of the title compound; ¹H-NMR (CDCl₃): d 2.57(s, 3H), 6.92(s,1H). The crude product was used in the next step without further purification.

### c) N-(3-Bromo-5-methyl-2-thienylsulfonyl)-N′-isopropylguanidine

A solution of 3-bromo-5-methylthiophene-2-sulfonyl chloride (1.1 g, 4.0 mmol) in ethyl ether (6 ml) was added to a stirred mixture of N-isopropylguanidine p-toluenesulfonate (1.31 g, 4.8 mmol) in 1N sodium hydroxide 9 ml and ethyl ether (12 ml), and the mixture was stirred at room temperature for 2 h. The solid, which precipitated was filtered off, washed with water and dried to give 0.81 g (59%) of the title compound; ¹H-NMR (DMSO-d₆): δ1.07 (d, 6H), 2.42 (s, 3H), 3.6-4.0 (very broad peaks, 1H), 6.4-7.5 (broad peaks, 3H), 6.89 (s, 1H). The crude product was used in the next step without further purification.

### d) 3-Isopropylamino-6-methyl-4H-thieno[3,2-e]-1,2,4-thiadiazine 1,1-dioxide

A mixture of N-(3-bromo-5-methyl-2-thienylsulfonyl)-N′-isopropylguanidine (0.68 g, 2.0 mmol), potassium carbonate (0.28g, 2.0 mmol) and copper bronze (25 mg) in dry N,N-dimethylformamide (5 ml) was stirred at 140° C. for 4 h under nitrogen. The cooled mixture was evaporated to dryness and the residue was stirred in a mixture of water (5 ml) and 1N sodium hydroxide (4 ml) followed by filtration. The filtrate was adjusted to pH<2 with 4M hydrochloric acid and the resulting solid was isolated by filtration, washed with water and recrystallized from ethanol to give 269 mg (52%) of the pure title compound; mp 272-273° C.; ¹H-NMR (DMSO-d₆): δ1.15 (d, 6H), 2.48 (s, 1H), 3.86 (m, 1H), 6.69 (s, 1H), 6.92 (br d, 1H), 10.59 (br s, 1H); MS: m/z 259 (M⁺); (C₉H₁₃N₃O₂S₂) calc. C, 41.68; H, 5.05; N, 16.20; found C, 41.69; H, 5.16; N, 16.06.

### EXAMPLE 30

### 6-Methyl-3-propylamino-4H-thieno[3,2-e]-1,2,4-thiadiazine 1,1-dioxide

### a) N-(3-Bromo-5-methyl-2-thienylsulfonyl)-N′-propylguanidine

A solution of 3-bromo-5-methylthiophene-2-sulfonyl chloride (0.65 g, 2.36 mmol) in ethyl ether (5 ml) was added to a stirred mixture of N-propylguanidine hydrochloride (0.39 g, 2.81 mmol) in 1N sodium hydroxide (5.3 ml) and ethyl ether (5 ml), and the mixture was stirred at room temperature for 2 h. The solid, which precipitated was filtered off, washed with ethyl ether and dried to give 0.53 g (66%) of the title compound; ¹H-NMR (DMSO-d₆): δ0.85 (t, 3H), 1.45 (sext, 2H), 2.43 (s, 3H), 3.06 (br s, 2H), 6.5-7.6 (broad peaks, 3H), 6.89 (s, 1H). The crude product was used in the next step without further purification.

### b) 6-Methyl-3-propylamino-4H-thieno[3,2-e]-1,2,4-thiadiazine 1,1-dioxide

A mixture of N-(3-bromo-5-methyl-2-thienylsulfonyl)-N′-propylguanidine (0.51 g, 1.5 mmol), potassium carbonate (0.21 g, 1.5 mmol) and copper bronze (25 mg) in dry N,N-dimethylformamide (5 ml) was stirred at 120° C. for 2 h under nitrogen. The cooled mixture was evaporated to dryness and the residue was stirred in hot 1N sodium hydroxide (10 ml), treated with decolorizing charcoal, and filtered. The filtrate was adjusted to pH<2 with 4M hydrochloric acid and the resulting solid was isolated by filtration, washed with water and recrystallized from ethanol to give 155 mg (40%) of the pure title compound; mp 263-264° C.; ¹H-NMR (DMSO-d₆): δ0.90 (t, 3H), 1.51 (sext, 2H), 2.49 (s, 2H), 3.14 (dt, 2H), 6.68 (s, 1), 7.05 (br t, 1H), 10.81 (br s, 1H); MS: m/z 259 (M⁺); (C₉H₁₃N₃O₂S₂) calc. C, 41.68; H, 5.05; N, 16.20; found C, 41.83; H, 5.05; N, 15.99.

### EXAMPLE 31

### 3-sec-Butylamino-6-methyl-4H-thieno[3,2-e]-1,2,4-thiadiazine 1,1-dioxide

### a) N-(3-Bromo-5-methyl-2-thienylsulfonyl)-N′-sec-butylguanidine

A solution of 3-bromo-5-methylthiophene-2-sulfonyl chloride (0.76 g, 2.75 mmol) in ethyl ether (5 ml) was added to a stirred mixture of N-sec-butylguanidine hydrochloride (0.50 g, 3.3 mmol) in 1N sodium hydroxide (6.2 ml) and ethyl ether (5 ml), and the mixture was stirred at room temperature for 1½ h. The solid, which precipitated was filtered off, washed with ethyl ether and dried to give 0.68 g (70%) of the title compound; ¹H-NMR (DMSO-d₆): δ0.81 (t, 3H), 1.04 (d, 3H), 1.40 (quint, 2H), 2.42 (s, 3H), 3.4-3.8 (broad peaks, 1H), 6.4-7.4 (broad peaks, 3H), 6.90 (s, 1H). The crude product was used in the next step without further purification.

### b) 3-sec-Butylamino-6-methyl-4H-thieno[3,2-e]-1,2,4-thiadiazine 1,1-dioxide

A mixture of N-(3-bromo-5-methyl-2-thienylsulfonyl)-N′-sec-butylguanidine (0.60 g, 1.65 mmol), potassium carbonate (0.23 g, 1.65 mmol) and copper bronze (40 mg) in dry N,N-dimethylformamide (5 ml) was stirred at 120° C. for 4 h under nitrogen. The cooled mixture was evaporated to dryness and the residue was stirred in hot 1N sodium hydroxide (12 ml), treated with decolorizing charcoal, and filtered. The filtrate was adjusted to pH<2 with 4M hydrochloric acid and the resulting solid was isolated by filtration, washed with water and recrystallized from ethanol to give 174 mg (39%) of the pure title compound; mp 224-225° C.; ¹H-NMR (DMSO-d₆): δ0.89 (t, 3H), 1.12 (d, 3H), 1.49 (m, 2H), 2.49 (s, 3H), 3.69 (m, 1H), 6.69 (s, 1H), 6.88 (br s, 1H), 10.59 (br s, 1H); MS: m/z 273 (M⁺); (C₁₀H₁₅N₃O₂S₂) calc. C, 43.94; H, 5.53; N, 15.37; found C, 43.62; H, 5.54; N, 15.15.

### EXAMPLE 32

### 6-chloro-3-(1-methylcyclopropyl)amino-4H-thieno[3,2-e]-1,2,4-thiadiazine 1,1-dioxide

### a) N-(3-Bromo-5-chloro-2-thienylsulfonyl)-N′-(1-methylcyclopropyl)guanidine

(1-Methylcyclopropyl)guanidine hydrochloride (2.9 g, 19.4 mmol), 2N sodium hydroxide (18 ml) and toluene (65 ml) were stirred until the (1-methylcyclopropyl)guanidine hydrochloride dissolved. Crude 3-bromo-5-chlorothiophene-2-sulfonyl chloride (4.2 g, 14.1 mmol) dissolved in toluene (20 ml) was added during 15 minutes at room temperature. The reaction mixture was stirred for 2 h until no starting material could be detected. The solid, which precipitated was isolated by filtration, giving the title compound (4.1 g, 78%); ¹H-NMR (DMSO) δ: 0.65 (br s, 4H), 1.3 (s, 3H), 7.32 (s, 1H), 7.9 (br s, 1H).

### b) 6-chloro-3-(1-methylcyclopropyl)amino-4H-thieno[3,2-e]-1,2,4-thiadiazine 1,1-dioxide

A mixture of N-(3-bromo-5-chloro-2-thienylsulfonyl)-N′-(1-methylcyclopropyl)guanidine (0.7 g, 1.88 mmol), cesium carbonate (0.92 g, 2.82 mmol) and copper(I) oxide (16 mg) in 1-butanol (10 ml) was stirred at 110° C. for 3 h under nitrogen. The reaction mixture was evaporated to dryness and the residue was treated with water (50 ml) and ethyl acetate (50 ml) and finally basified with 1N sodium hydroxide to pH 10. The phases were separated, and the aqueous phase was extracted with ethyl acetate. The organic phases were combined, and the solvent removed under reduced pressure, giving the title compound (0.16 g, 30%); mp 258° C.; ¹H-NMR (DMSO-d₆): δ0.67 (m, 4H), 1.33 (s, 3H), 7.11 (br s, 1H), 7.89 (br s, 1H), 11.25 (br s, 1H).

### EXAMPLE 33

### 6-chloro-3-(1-methylcyclopropyl)amino-4H-thieno[3,2-e]-1,2,4-thiadiazine 1,1-dioxidea) N-(3,5-dichloro-2-thienylsulfonyl)-N′-(1-methylcyclopropyl)guanidine

(1-Methylcyclopropyl)guanidine hydrochloride (20.0 g, 133.7 mmol), 2N sodium hydroxide (95 ml) and toluene (150 ml) were stirred until the (1-methylcyclopropyl)guanidine hydrochloride dissolved. Crude 3,5-dichlorothiophene-2-sulfonyl chloride (10.0 g, 39.8 mmol) dissolved in toluene (50 ml) was slowly added during 90 min at room temperature. The reaction mixture was stirred overnight. The solid was isolated by filtration giving (5.4 g, 41%) of the title compound. The organic phase of the filtrate was separated, dried over Na₂SO₄ and the solvent removed under reduced pressure, giving a second crop of the title compound (2.8 g, 22%). ¹H-NMR (DMSO) δ: 0.65 (br s, 4H), 1.25 (s, 3H), 6.6 (br s, 1H), 7.20 (br s, 1H), 7.32 (s, 1H), 7.95 (br s, 1H).

### b) 6-chloro-3-(1-methylcyclopropyl)amino-4H-thieno[3,2-e]-1,2,4-thiadiazine 1,1-dioxide

A mixture of N-(3,5-dichloro-2-thienylsulfonyl)-N′-(1-methylcyclopropyl)guanidine (0.87 g, 2.6 mmol), cesium carbonate (1.27 g, 3.9 mmol) and copper(I) oxide (23 mg) in 1-butanol (18 ml) was stirred at 115° C. for 24 h under nitrogen. The reaction mixture was evaporated to dryness and the residue was treated with 20 ml of water, 30 ml of EtOAc and 1 M HCI to pH 6. The phases were separated. The organic phase was washed with brine, dried over Na₂SO₄, and the solvent removed under reduced pressure, giving the title compound (0.51 g, 67%); mp 258° C.; ¹H-NMR (DMSO-d₆): δ0.67 (m, 4H), 1.33 (s, 3H), 7.11 (br s, 1H), 7.89 (br s, 1H), 11.25 (br s, 1H).

What is claimed is:

1. A process for the preparation of a compound of formula (I) 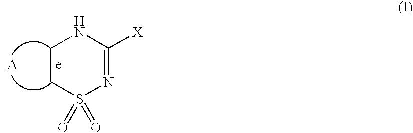
wherein
X is NR²R³, SR¹, S(═O)R¹, S(═O)₂R¹ or OR¹;
R¹ is hydrogen; C₃₋₆-cycloalkyl or (C₃₋₆-cycloalkyl)C₁₋₆-alkyl, wherein the C₃₋₆-cycloalkyl group is optionally mono- or polysubstituted with C₁₋₆-alkyl, halogen, hydroxy or C₁₋₆-alkoxy; a 3-6 membered saturated ring system comprising one or more nitrogen-, oxygen- or sulfur atoms, optionally being mono- or polysubstituted with halogen, cyano, trifluoromethyl, C₁₋₆-alkyl, C₁₋₆-alkoxy, C₁₋₆-alkoxy-C₁₋₆-alkyl, aryl, arylalkyl, hydroxy, oxo, nitro, amino, C₁₋₆-monoalkyl or dialkylamino; straight or branched C₁₋₁₈-alkyl, C₂₋₁₈-alkenyl or C₂₋₁₈-alkynyl, wherein each of the groups is optionally mono- or polysubstituted with halogen, hydroxy, C₁₋₆-alkoxy, C₁₋₆-alkylthio, C₃₋₆-cycloalkyl, nitro, amino, C₁₋₆-monoalkyl- or dialkylamino, cyano, oxo, formyl, acyl, carboxy, C₁₋₆-alkoxycarbonyl, carbamoyl, formylamino, C₁₋₆-alkylcarbonylamino, aryl, aryloxy, arylalkoxy; or bicycloalkyl, aryl, heteroaryl, arylalkyl or heteroarylalkyl, wherein each of the groups is optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;
R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or polysubstituted with halogen;
R³ is hydrogen; C₃₋₆-cycloalkyl or (C₃₋₆-cycloalkyl)C₁₋₆-alkyl, wherein the C₃₋₆-cycloalkyl group is optionally mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl; or
R³ is —OR⁴; —C(═Z)R⁴; —NR⁴R⁵; or bicycloalkyl, aryl, heteroaryl, arylalkyl or heteroarylalkyl, optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;
R⁴ is hydrogen; C₃₋₆-cycloalkyl or (C₃₋₆-cycloalkyl)C₁₋₆-alkyl, wherein the C₃₋₆-cycloalkyl group is optionally mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
Z is O or S;
R⁵ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or
when R³ is —NR⁴R⁵, R⁴ and R⁵ together with the nitrogen atom form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, or oxo; or
when X is —NR²R³, R² and R³ together with the nitrogen atom form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;
A together with the carbon atoms forming bond e of formula I represents a 5 membered heterocyclic system comprising one or more nitrogen-, oxygen- or sulfur atoms, the heterocyclic system optionally being mono- or polysubstituted with halogen; C₁₋₁₈-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, aryl, arylalkyl, or aryloxy, wherein the aryl group is optionally mono- or polysubstituted with C₁₋₆-alkyl, perhalomethyl, halogen, hydroxy or C₁₋₆-alkoxy; C₁₋₆-alkoxycarbonyl; C₁₋₆-alkoxycarbonyl-C₁₋₆-alkyl; carbamyl; carbamylmethyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino; thiocarbamyl; thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; formyl; or a 5-6 membered nitrogen, oxygen or sulfur containing ring, optionally substituted with C₁₋₆-alkyl or phenyl, wherein the phenyl group is optionally mono- or polysubstituted with C₁₋₆-alkyl, perhalomethyl, halogen, hydroxy or C₁₋₆-alkoxy; or
a salt thereof with a pharmaceutically acceptable acid or base, or an optical isomer thereof, or a tautomeric form thereof, or metabolites or prodrugs thereof, comprising one of the following methods:
a) reacting a compound of formula (II) 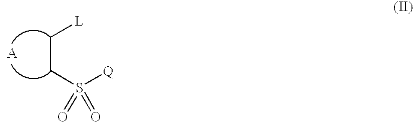
wherein A is as defined above, L is a leaving group selected from alkoxy, alkylthio, alkylsulfinyl, alkylsulfonyl, nitro or halogen and Q is halogen, with a compound of formula (III), 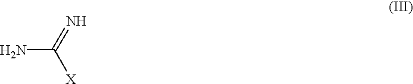
wherein X is NR²R³, wherein R² and R³ are defined above, or a suitable salt thereof, in the presence of a base in solvent 1, to form a compound of formula (IV) 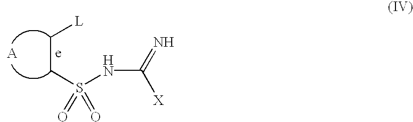
wherein A, L and X are as defined above, and cyclizing the compound of formula (IV) in solvent 2, optionally in the presence of a base, and optionally with a metal catalyst, to form a compound of formula (I), or
b) reacting a compound of formula (II) 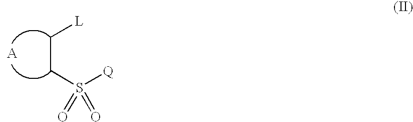
wherein A is as defined above, L is a leaving group selected from alkoxy, alkylthio, alkylsulfinyl, alkylsulfonyl or halogen and Q is halogen, with a compound of formula (III) 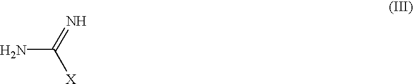
wherein X is SR¹, S(═O)R¹ or S(═O)₂R¹, wherein R¹ is defined above, or a suitable salt thereof, in the presence of a base in solvent 1, to form a compound of formula (IV) 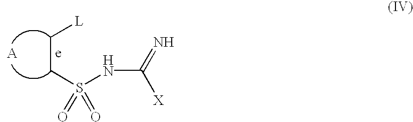
wherein A, L and X are as defined above, and cyclizing the compound of formula (IV) in solvent 2, optionally in the presence of a base, and optionally with a metal catalyst, to form a compound of formula (I), or
c) reacting a compound of formula (II) 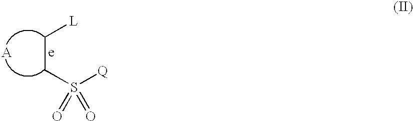
wherein A is as defined above, L is a leaving group selected from alkoxy, alkylthio, alkylsulfinyl, alkylsulfonyl or halogen and Q is halogen, with a compound of formula (III), 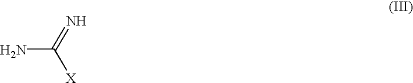
wherein X is OR¹, wherein R¹ is defined above, or a suitable salt thereof, in the presence of a base in solvent 1, to form a compound of formula (IV) 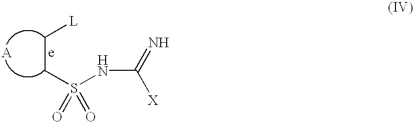
wherein A, L and X are as defined above, and cyclizing the compound of formula (IV) in solvent 2, optionally in the presence of a base, and optionally with a metal catalyst, to form a compound of formula (I), or
d) transforming a compound of formula (IV) to a compound of formula (IV′) 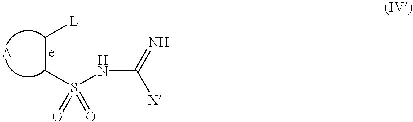
wherein A, L and X are as defined above, and X is transformed into X′, wherein X′ is selected from the groups defined for X, with the proviso that X′≠X, and cyclizing the compound of formula (IV′) in solvent 2, optionally in the presence of a base, and optionally with a metal catalyst, to form a compound of formula (I), or
e) transforming a compound of formula (I), prepared as described above, by oxidation or substitution or both, to form another compound of formula (I).

2. A process according to claim 1 comprising:
reacting a compound of formula (II) 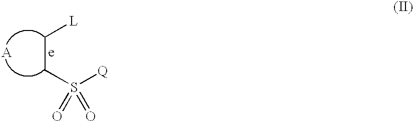
wherein A is as defined above, L is a leaving group selected from alkoxy, alkylthio, alkylsulfinyl, alkylsulfonyl, nitro or halogen and Q is halogen, with a compound of formula (III) 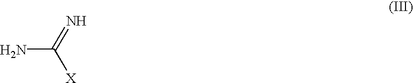
wherein X is NR²R³, wherein R² and R³ are defined above, or a suitable salt thereof, in the presence of a base in solvent 1, to form a compound of formula (IV) 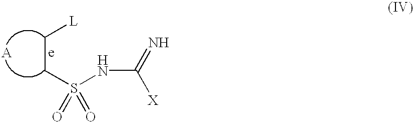
wherein A, L and X are as defined above, and cyclizing the compound of formula (IV) in solvent 2, optionally in the presence of a base, and optionally by treatment with a metal catalyst, to form a compound of formula (I).

3. A process according to claim 1 comprising:
reacting a compound of formula (II) 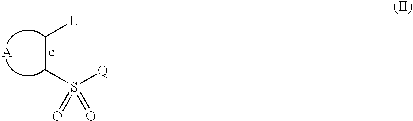
wherein A is as defined above, L is a leaving group selected from alkoxy, alkylthio, alkylsulfinyl, alkylsulfonyl, nitro or halogen and Q is halogen, with a compound of formula (III), 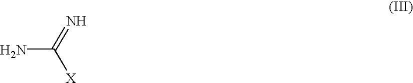
wherein X is SR¹, S(═O)R¹ or S(═O)₂R¹, wherein R¹ is defined above, or a suitable salt thereof, in the presence of a base in solvent 1, to form a compound of formula (IV) 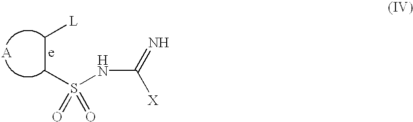
wherein A, L and X are as defined above, and cyclizing the compound of formula (IV) in solvent 2, optionally in the presence of a base and, optionally by treatment with a metal catalyst, to form a compound of formula (I).

4. A process according to claim 1 comprising:
reacting a compound of formula (II) 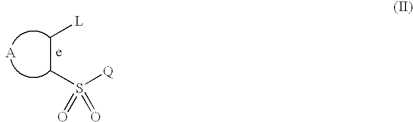
wherein A is as defined above, L is a leaving group selected from alkoxy, alkylthio, alkylsulfinyl, alkylsulfonyl, nitro or halogen and Q is halogen, with a compound of formula (III), 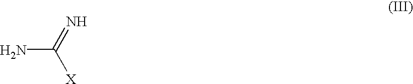
wherein X is OR¹, wherein R¹ is defined above, or a suitable salt thereof, in the presence of a base in solvent 1, to form a compound of formula (IV) 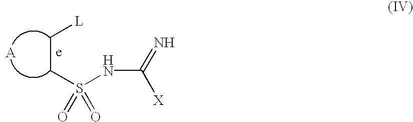
wherein A, L and X are as defined above, and cyclizing the compound of formula (IV) in solvent 2, optionally in the presence of a base and, optionally by treatment with a metal catalyst, to form a compound of formula (I).

5. A process according to claim 1 comprising:
transforming a compound of formula (IV) into a compound of formula (IV′) 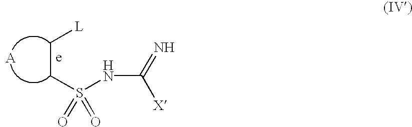
wherein A, L and X are as defined above, and X is transformed into X′, wherein X′ is selected from the groups defined for X, with the proviso that X′≠X, and cyclizing the compound of formula (IV′) in solvent 2, optionally in the presence of a base and, optionally by treatment with a metal catalyst, to form a compound of formula (I).

6. A process according to claim 1 comprising:
transforming a compound of formula (IV) 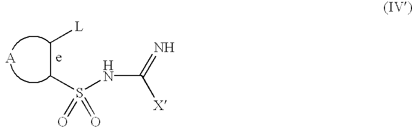
wherein A, and L are as defined above and X is SR¹, S(═O)R¹ or S(═O)₂R¹, wherein R¹ is defined above, into a compound of formula (V) 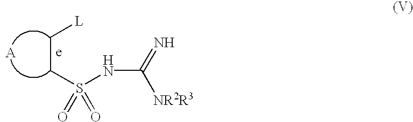
wherein A, L and R² and R³ are as defined above, and cyclizing the compound of formula (V) in solvent 2, optionally in the presence of a base and, optionally by treatment with a metal catalyst, to form a compound of formula (I).

7. A process according to claim 1, wherein the process further comprises cyclizing the compound of formula (IV) in solvent 2 in the presence of a base.

8. A process according to claim 1, wherein the process further comprises cyclizing the compound of formula (IV) in solvent 2 in the presence of a base and by treatment with a metal catalyst.

9. A process according to claim 1, wherein the process further comprises cyclizing the compound of formula (IV) in solvent 2 in the presence of a base and without a metal catalyst.

10. A process according claim 1, wherein the process further comprises cyclizing the compound of formula (IV) in solvent 2 without the presence of a base.

11. A process according to claim 1, wherein the process further comprises cyclizing the compound of formula (IV) in solvent 2 by treatment with a metal catalyst without the presence of a base.

12. A process according to claim 1, wherein the process further comprises cyclizing the compound of formula (IV) in solvent 2 without the presence of a base and without a metal catalyst.

13. A process according to claim 1, wherein the process further comprises transforming a compound of formula (I), prepared as described above, by oxidation or substitution or both, to form another compound of formula (I).

14. A process according to claim 1, wherein the base is selected from sodium hydroxide, potassium carbonate, cesium carbonate or potassium hydroxide.

15. A process according to claim 1, wherein solvent 1 is selected from diethyl ether, acetone, toluene or t-butyl-methyl ether.

16. A process according to claim 1, wherein solvent 2 is selected from N,N-dimethylformamide, toluene, xylene, 1-butanol, N-methyl-2-pyrrolidinone, sulfolane, dimethylsulfoxide, DMPU or water.

17. A process according to claim 1, wherein the metal catalyst is selected from copper bronze, copper oxide, copper chloride, copper bromide or copper iodide.

18. A compound selected from the group consisting of:
3-Amino-6-chloro-4H-thieno[3,2-e]-1,2,4-thiadiazine 1,1-dioxide;
7-Bromo-6-chloro-3-propylaminothieno[2,3-e]-1,2,4-thiadiazine 1,1-dioxide;
7-Bromo-3-(sec-butylamino)-6-chloro-4H-thieno[2,3-e]-1,2,4-thiadiazine 1,1-dioxide;
7-Bromo-6-chloro-3-cyclobutylamino-4H-thieno[2,3-e]-1,2,4-thiadiazine 1,1-dioxide;
6-Chloro-3-methylsulfanyl-4H-thieno[2,3-e]-1,2,4-thiadiazine 1,1-dioxide; or
6-Chloro-3-methylsulfinyl-4H-thieno[2,3-e]-1,2,4-thiadiazine 1,1-dioxide+
obtained by a process according to claim 1.

19. A compound selected from the group consisting of:
6-Bromo-3-methylsulfanyl-4H-thieno[2,3-e]-1,2,4-thiadiazine 1,1-dioxide; 6-Bromo-3-methylsulfinyl-4H-thieno[2,3-e]-1,2,4-thiadiazine 1,1-dioxide; 3-Amino-6-bromo-4H-thieno[3,2-e]-1,2,4-thiadiazine 1,1-dioxide; 6-Chloro-3-ethylamino-4H-thieno[2,3-e]-1,2,4-thiadiazine 1,1-dioxide;
6-Chloro-3-propylamino-4H-thieno[2,3-e]-1,2,4-thiadiazine 1,1-dioxide;
3-Isopropylamino-6-methyl-4H-thieno[3,2-e]-1,2,4-thiadiazine 1,1-dioxide;
6-Methyl-3-propylamino-4H-thieno[3,2-e]-1,2,4-thiadiazine 1,1-dioxide; or
3-sec-Butylamino-6-methyl-4H-thieno[3,2-e]-1,2,4-thiadiazine 1,1-dioxide
obtained by a process according to claim 1.

20. A pharmaceutical composition for the treatment or prophylaxis of Type I or Type II diabetes comprising a compound according to claim 18 and a pharmaceutically acceptable carrier.

21. A pharmaceutical composition for the treatment or prophylaxis of Type I or Type II diabetes comprising a compound according to claim 19 and a pharmaceutically acceptable carrier.

22. A method of treating Type I or Type II diabetes which comprises administering an effective or prophylactic amount of a compound according to claim 18 to a person suffering from Type I or Type II diabetes.

23. A method of treating Type I or Type II diabetes which comprises administering an effective or prophylactic amount of a compound according to claim 19 to a person suffering from Type I or Type II diabetes.
